(19) United States
(12) Patent Application Publication
(10) Pub. No.: US 2022/0105019 A1
(43) Pub. Date: Apr. 7, 2022

(54) CITRUS FIBERS AND SCLEROGLUCAN COMPOSITIONS AND THE USE THEREOF IN PERSONAL CARE APPLICATIONS

(71) Applicant: CARGILL, INCORPORATED, Wayzala, MN (US)

(72) Inventors: Emmanuel Paul Jos Marie EVERAERT, Galder (NL); Jacques André Christian MAZOYER, Donville les Bains (FR)

(73) Assignee: CARGILL, INCORPORATED, Wayzata, MN (US)

(21) Appl. No.: 17/427,707

(22) PCT Filed: Feb. 3, 2020
(86) PCT No.: PCT/US2020/016413

§ 371 Date: Aug. 2, 2021

Related U.S. Application Data
(60) Provisional application No. 62/799,962, filed on Feb. 1, 2019.
(60) Provisional application No. 62/822,359, filed on Mar. 22, 2019.

(51) Int. Cl. A61K 8/73; A61K 8/02; A61K 8/9789; A61Q 5/02; A61Q 19/00

(52) U.S. Cl. CPC A61K 8/73; A61K 8/027; A61Q 19/00; A61Q 5/02; A61K 8/9789

(57) ABSTRACT

The invention relates to compositions comprising citrus fibers and 1,3-β-D-glucans, specifically scleroglucan, for use in the manufacture emulsions or aqueous mixtures for use in topical formulations. The invention further relates to processes for manufacturing an emulsion, or aqueous mixture, or topical formulation and to uses thereof, especially in personal care products.

### CROSS-REFERENCE TO RELATED APPLICATIONS

This application claims the benefit of U.S. Provisional Patent Application No. 62/799,962, filed Feb. 1, 2019, entitled CITRUS FIBERS AND SCLEROGLUCAN EMULSIONS AND THE USE THEREOF IN PERSONAL CARE APPLICATIONS, and U.S. Provisional Patent Application No. 62/822,359, filed Mar. 22, 2019, entitled CITRUS FIBERS AND SCLEROGLUCAN EMULSIONS AND THE USE THEREOF IN PERSONAL CARE APPLICATIONS, each of which are hereby incorporated by reference herein in its entirety.

### FIELD OF INVENTION

The invention relates to blends of citrus fibers and a 1,3-β-D-glucan, the use of these blends in creating aqueous solutions or emulsions, and for their use in the manufacture of topical formulations for personal care.

### BACKGROUND

Citrus fibers (e.g. citrus peel fibres or “CPF”) have been used as ingredients in a variety of products intended for personal care (e.g. topical formulations). Citrus fibers are derived from the cell walls of citrus fruits and comprise microfibrils of cellulose. Common varieties of the citrus fruit include oranges, sweet oranges, clementines, kumquats, tangerines, tangelos, satsumas, mandarins, grapefruits, citrons, pomelos, lemons, rough lemons, limes and leech limes.

Citrus fiber is to be distinguished from citrus pulp, which are whole juice sacs and are sometimes referred to as citrus vesicles, coarse pulp, floaters, citrus cells, floating pulp, juice sacs, or pulp. Citrus fiber is also to be distinguished from citrus rag, which is the stringy central portion and membranous walls of a citrus fruit.

Citrus fibers are typically obtained from a source of citrus fibers, e.g. citrus peel, citrus pulp, citrus rag or combinations thereof. Moreover, the citrus fibers contain the components of the primary cell walls of the citrus fruit such as cellulose, pectin and hemicelluloses and may also contain proteins.

Using various techniques, such as the ones disclosed in WO2012/016201 and WO2018/009749, the properties of citrus fibers can be tailored to impart specific rheological behaviours, texture, and appearance to the final product.

Compositions containing citrus fibers and additional compounds, such as: sugars, proteins, polysaccharides, polyol, glucose, sucrose, glycerol and sorbitol, are known in the art, for example in WO 2017/023722 and WO 2017/019752.

However, conventional formulations containing citrus fibers can have sub-optimal characteristics. For instance, they may be non-white (e.g. grey or beige) in colour with an unpleasant (e.g. jelly, stringy or lumpy) texture and/or consistency. They may also have low stability and/or high viscosity. Such characteristics may deleteriously influence the sensory appeal of the product to the consumer and may cause problems during processing.

There is an unmet need for a formulation comprising citrus fibers which has optimal sensory characteristics. In particular, a need exists for formulations with optimum stability, viscosity, colour, texture, and/or consistency. These formulations can advantageously be utilized in a variety of end use applications including personal care compositions of any type.

### SUMMARY OF INVENTION

The present invention seeks to address the problems identified above by providing compositions and emulsions comprising an aqueous phase and an oil phase, the emulsion comprising citrus fibers and a 1,3-β-D-glucan. The emulsion is preferably and oil-in-water emulsion.

The citrus fibers and 1,3-β-D-glucan are preferably in a ratio of between 99:1 and 1:99. For example, the ratio of citrus fibers and 1,3-β-D-glucan may be between may be between 90:10 and 10:90, or between 80:20 and 20:80, or between 70:30 and 30:70, or between 60:40 and 40:60, or approximately 50:50.

The amount of the aqueous phase in the emulsion may be between 30 wt % to 99 wt. For example, the amount of the aqueous phase in the emulsion may be between 40 wt % to 60 wt %, preferably between 60 wt % to 80 wt %, most preferably between 70 wt % to 90 wt %.

The amount of the oil phase in the emulsion may be between 0.1 wt % to 70%. For example, the amount of the oil phase in the emulsion may be between 5 wt % to 55 wt %, preferably between 10 wt % and 40 wt %, most preferably between 10 wt % and 30 wt %.

The citrus fibers and/or 1,3-β-D-glucan may be dispersed, and thus present, in the aqueous phase of the emulsion. The citrus fibers and/or 1,3-β-D-glucan may be dispersed, and thus present, in the oil phase of the emulsion. The citrus fibers and/or 1,3-β-D-glucan may be dispersed, and thus present, in the oil phase and the aqueous phase of the emulsion.

In one aspect of the invention, the oil phase of the emulsion comprises a natural oil, hydrogenated natural oil, synthetic oils, or petroleum oils.

The emulsion may further comprise at least one further ingredient. The further ingredient may include, without limitation, a preservative, salt, vitamin, emulsifier, texturiser, nutrient, micronutrient, sugar, protein, polysaccharide, polyol, glucose, sucrose, glycerol, sorbitol, pH adjusters, emollients, dyes, pigments, skin actives, waxes, or silicones.

The citrus fibers and 1,3-β-D-glucan when used to manufacture the emulsion may be in the form of a blend. Such a blend can be prepared by the mechanical mixing of citrus fibers and 1,3-β-D-glucan or alternatively the blend can be prepared by the co-drying or co-processing of citrus fibers and 1,3-β-D-glucan. Preferably, the citrus fibers and 1,3-β-D-glucan when used to manufacture the emulsion are in form of a co-processed blend.

The amount of the blend in the emulsion may be between 0.1 wt % to 5 wt % relative to the total weight of the emulsion. For example, the amount of the blend in the emulsion may be between 0.1 wt % to 4 wt %, preferably between 0.2 wt % and 3 wt %, most preferably between 0.3 wt % and 3 wt %.

The invention also provides a topical formulation comprising the emulsion disclosed herein.

The invention further provides a dry blend comprising citrus fibers and a 1,3-β-D-glucan. The dry blend comprising citrus fibers and 1,3-β-D-glucan may be a co-dryed or co-processed blend.

The concentration of citrus fibers in the blend may be between 25 wt % and 99 wt % relative to the total weight of the blend. For example, the concentration of citrus fibers may be between 35 wt % to 95 wt %, preferably between 45 wt % and 90 wt %, most preferably between 55 wt % and 85 wt %.

The concentration of 1,3-β-D-glucan in the blend may be between 0.1 wt % and 75 wt % relative to the total weight of the blend. For example, the concentration of 1,3-β-D-glucan may be between 1 wt % to 65 wt %, preferably between 10 wt % and 55 wt %, most preferably between 15 wt % and 45 wt %.

The invention also provides an aqueous mixture comprising the dry blend disclosed herein. The aqueous mixture may have a viscosity from 3 to 300,000 Cps.

The invention further provides a process for producing an emulsion of citrus fibers and a 1,3-β-D-glucan as disclosed herein. The process comprises the steps of:

a) providing a blend of citrus fibers and a 1,3-β-D-glucan;
b) adding the blend to water and mixing to form an aqueous phase;
c) dispersing oil in the aqueous phase to obtain an emulsion. Optionally, a homogenization step may be included before and/or after step c).

Preferably, the 1,3-β-D-glucan used in the invention is scleroglucan.

### DETAILED DESCRIPTION

Explanations of abbreviations and terms used in this disclosure are provided to assist in comprehending and practicing the invention.

All ratios of emulsion or formulation components refer to percentage by weight (wt %), unless otherwise specified.

All parameter ranges disclosed include the end-points and all values in between, unless otherwise specified.

Representative features are set out in the following description, which stand alone or may be combined, in any combination, with one or more features disclosed elsewhere in the description and/or drawings of the specification.

When used in this specification and claims, the terms “comprises” and “comprising” and variations thereof mean that the specified features, steps or integers are included. The terms are not to be interpreted to exclude the presence of other features, steps or components.

### Citrus Fibers

The term “citrus fibers” as used herein, refers to an elongated object obtained from citrus fruit and comprising microfibrils of cellulose. Citrus fibers useful in the present disclosure can be prepared for example by the methods of WO 2013/109721. The citrus fibers typically has a length (major axis) and a width (minor axis) with a length to width ratio of at least 5, more preferably at least 10, or most preferably at least 15, as observed and measured by a high-resolution scanning electron microscope (“SEM”). The length of the citrus fibers is preferably at least 0.5 μm, more preferably at least 1 μm. The width of the citrus fibers is preferably at most 100 nm, more preferably at most 50 nm, most preferably at most 15 nm. The microfibrils forming a citrus fiber, typically have a length of between 1 μm and 500 μm. The length of the majority (i.e. at least 75%) of the microfibrils forming a citrus fibers is typically at most 250 μm or at most 100 μm. Preferrably, the citrus fibers have a particle size between 1 μm and 250 μm. Sample of citrus fibers may be milled and sieved according to any method known in the art to yield citrus fibers of various particle sizes.

The citrus fibers used in the invention may have undergone substantial chemical modification, i.e. said fibers may be subjected to chemical modification processes, such as any of esterification, derivatisation or enzymatic modification and combinations thereof. Preferrably the citrus fibers used in the invention have not undergone substantial chemical modification.

### 1,3-β-D-glucan

1,3-β-D-glucan is a polysaccharide characterized by a backbone of D-glucose residues linked in β-1,3-(1,3) fashion, wherein the different 1,3-β-D-glucans structurally differ from each other in terms of their side groups and molecular weights. Curdlan for example, consists exclusively of the β-1,3-(1,3)-D glucose backbone, whereas schizophyllan, scleroglucan and yeast glucan contain β-(1,6)-glycosyl side chains. 1,3-β-D-glucan are typically produced by microbial fermentation, the fermentation broth being used directly or in diluted or purified form, usually after having been pasteurized (see e.g. U.S. Pat. No. 3,301,848). The 1,3-β-D-glucan can be used in purified form or as a mixture of 1,3-β-D-glucan and fermentation residuals. For the purpose of the invention, the 1,3-β-D-glucan used is preferably purified to reduce and neutralize the amount and activity of microbial cells and/or water-soluble constituents of the fermentation broth other than the 1,3-β-D-glucan. WO 2009/062561 discloses a method of manufacturing such high purity 1,3-β-D-glucans.

The 1,3-β-D-glucans used within the present invention include any polysaccharides classified as 1,3-β-D-glucans, i.e. any polysaccharide which has β-(1,3)-linked backbone of D-glucose residues. Examples of such 1,3-β-D-glucan s include curdlan (a homopolymer of β-(1,3)-linked D-glucose residues produced from, e.g., Agrobacterium spp.), grifolan (a branched 1,3-β-D-glucan produced from, e.g., the fungus Grifola frondosa), lentinan (a branched 1,3-β-D-glucan having two glucose branches attached at each fifth glucose residue of β-(1,3)-backbone produces from, e.g., the fungus Lentinus eeodes), schizophyllan (a branched 1,3-β-D-glucan having one glucose branch for every third glucose residue in the β-(1,3)-backbone produced from, e.g., the fungus Schizophyllan commune), scleroglucan (a branched 1,3-β-D-glucan with one out of three glucose molecules of the β-(1,3)-backbone being linked to a side D-glucose unit by a (1,6)-n bond produced from, e.g., fungi of the Sclerotium spp.), SSG (a highly branched β-(1,3)-glucan produced from, e.g., the fungus Sclerotinia sclerotiorum), soluble glucans from yeast (a 1,3-β-D-glucan with β-(1-6)-linked side groups produced from, e.g., Saccharomyces cerevisiae), and laminarin (a 1,3-β-D-glucan with β-(1,3)-glucan and β-(1,6)-glucan side groups produced from, e.g., the brown algae Laminaria digitata).

Preferably, the 1,3-β-D-glucan is scleroglucan.

Scleroglucan is a natural polysaccharide produced by fermentation of the filamentous fungus Sclerotium rolfsii. Its chemical structure consists of a linear β (1-3) D-glucose backbone with one β (1-6) D-glucose side chain every three main residues, as shown below:

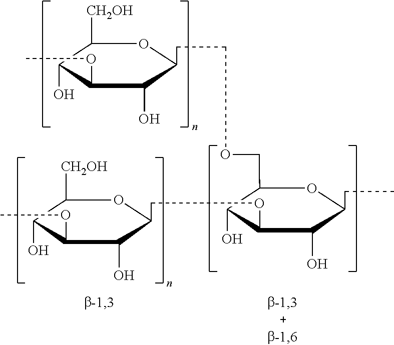

Scleroglucan exhibits high potential for commercialization and may show different branching frequency, side-chain length, and/or molecular weight depending on the producing strain or culture conditions. As used herein, the term “molecular weight” refers to the measure of the sum of the atomic weight values of the atoms in a molecule.

When the 1,3-β-D-glucan used is scleroglucan, it may be in a purified form or in a mix with fermentation residuals. Scleroglucan may be in the form of native scleroglucan or hydrolysed scleroglucan. The molecular weight of the scleroglucan may be between 50,000 and 6,000,000 Daltons. Preferably, if the scleroglucan is native scleroglucan, the molecular weight of the scleroglucan may be between 1,500,000 and 6,000,000 Daltons. Preferably, if the scleroglucan is a hydrolysed scleroglucan, the molecular weight may be between 50,000 and 100,000 Dalton.

Scleroglucan is able to from a triple helix in solution making it an ideal nature-derived thickener and stabilizer for formulations with smooth and soft textures. Water-solubility, viscosifying ability and wide stability over temperature, pH and salinity make scleroglucan useful for different personal care applications.

### Emulsion

In one aspect, the present invention is an oil-in-water emulsion. An emulsion may be defined as a mixture containing two immiscible liquids, in which one liquid is dispersed as droplets or globules throughout the other. The dispersed liquid is called the dispersed phase, while the other liquid is called the continuous phase. In an oil-in-water emulsion, as in the present invention, the oil is the dispersed phase or oil phase, and water is the continuous phase or aqueous phase.

The emulsion of the present invention comprises citrus fibers and a 1,3-β-D-glucan. Preferably, the 1,3-β-D-glucan is scleroglucan.

The citrus fibers and 1,3-β-D-glucan are preferably in a ratio of between 99:1 and 1:99. For example, the ratio of citrus fibers and 1,3-β-D-glucan may be between may be between 90:10 and 10:90, or between 80:20 and 20:80, or between 70:30 and 30:70, or between 60:40 and 40:60, or 50:50.

The representative emulsions have good stability, with little or no separation between the aqueous phase and oil phase over an extended period of time (e.g. 12 weeks) when measured at room temperature and/or elevated storage temperatures (e.g. 45° C.). Therefore, it may be used to make products (e.g. topical formulations) requiring a long shelf life.

### 1. Aqueous Phase

The inventive emulsion contains an aqueous phase. The aqueous phase may comprise or consist of water, in particular a demineralized water; a floral water such as cornflower water; a mineral water such as Vittel water, Lucas water or La Roche Posay water; and/or a spring water. Preferably, demineralized water is used as the aqueous phase utilized by the present invention.

The amount of the aqueous phase in the emulsion may be between 30 wt % to 99 wt %. For example, the amount of the aqueous phase in the emulsion may be between 40 wt % to 60 wt %, preferably between 60 wt % to 80 wt %, most preferably between 70 wt % to 90 wt %.

### 2. Oil Phase

The representative emulsions also contain an oil phase dispersed in the aqueous phase. As used herein, the term “dispersion” refers to an oil phase forming droplets inside the aqueous phase. The droplets may be of any size or shape. Preferably, the droplets are homogeneously distributed throughout the aqueous phase. The nature of the oil phase of the emulsion is not critical. The oil phase may thus consist of any fatty substance conventionally used in the cosmetic or dermatological fields; in particular the oil phase may preferably comprise at least one oil, i.e. any fatty substance that is in liquid form at room temperature (20-25° C.) and at atmospheric pressure (760 mmHg).

The preferred oil phase(s) comprises at least one oil which can be a hydrocarbon-based oil, i.e. an oil mainly containing hydrogen and carbon atoms and optionally oxygen, nitrogen, sulfur and/or phosphorus atoms, for example in the form of hydroxyl or acid radicals; a silicone oil, i.e. an oil comprising at least one silicon atom and preferably at least one Si-O group; a fluoro oil, i.e. an oil comprising at least one fluorine atom; a non-fluoro oil, or a mixture thereof. Preferably, the inventive emulsion comprises at least one hydrocarbon-based oil as the oil phase.

The hydrocarbon-based oils may be of animal origin or of vegetable origin, such as liquid triglycerides of fatty acids comprising from 4 to 20 carbon atoms, examples include, coconut oil, canola oil, rapeseed oil, sunflower oil; maize oil; soybean oil; cucumber oil; grape seed oil; sesame seed oil; hazelnut oil; apricot oil; macadamia oil; arara oil; castor oil; cocoa butter; almond oil; avocado oil; babassu oil; caprylic/capric acid triglycerides, such as those sold by Stearineries Dubois or those sold under the names Miglyol 810, 812 and 818 by Dynamit Nobel; Simmondsia Chinensis (Jojoba) Seed oil sold under the tradename Jojoba Oil Golden by Desert Whale; Beta-carotene sold under the tradename Betatene 30% OLV by Cognis (BASF); Rosa Canina Fruit Oil sold under the tradename Rosehip Seed Oil by Nestle World Trade Co.; shea butter oil; and mixtures thereof.

Preferably, the oil phase contains a vegetable oil and/or a vegetable fat; more preferably it contains coconut oil, more preferably it contains cocoa butter and a vegetable oil, e.g. almond oil; even more preferably, the oil phase contains caprylic/capric acid triglycerides, cocoa butter and a vegetable oil different that said triglycerides, e.g. almond oil.

The hydrocarbon-based oils may be linear or branched hydrocarbons of mineral or synthetic origin. Alternatively, the hydrocarbon-based oils may be synthetic ethers; synthetic esters; fatty alcohols that are liquid at room temperature, with a branched and/or unsaturated carbon-based chain containing from 12 to 26 carbon atoms; C12-C22 higher fatty acids; or mixtures thereof.

The amount of the oil phase in the emulsion may be between 0.1 wt % to 70%. For example, the amount of oil phase in the emulsion may be between 5 wt % to 55 wt %, preferably between 10 wt % and 40 wt %, most preferably between 10 wt % and 30 wt %.

### Blend

Advantageously, the emulsion of the invention is manufactured using a blend comprising or consisting of citrus fibres and 1,3-β-D-glucan. As used herein, the term “blend” refers to a mixture of two or more substances.

The blend may be a dry blend, meaning that it contains an amount of liquid, e.g. water and/or organic solvent, of less than 20 wt % relative to the total weight of the fibers. Preferably said fibers contain an amount of water (i.e. moisture content) relative to the total weight of the fibers of at most 12 wt %, more preferably at most 10 wt %, or most preferably at most 8 wt %. Such dry blends are economical to transport and store while being readily dispersible in the aqueous medium. The dry blend need not be completely dry or absent of water and/or organic solvent, therefore, the dry blend may contain between 0.5% and 20% water or any range in between including 0.5% and 12%, 0.5% and 8%, 1% and 12%, 1% and 10%, or 1% and 8%.

The amount of the blend in the emulsion may be between 0.1 wt % to 5 wt %. For example, the amount of the blend in the emulsion may be between 0.1 wt % to 4 wt %, preferably between 0.2 wt % and 3 wt %, most preferably between 0.3 wt % and 3 wt %.

The blend may be produced by mixing, co-drying, or by co-processing the citrus fibers and 1,3-β-D-glucan. Most preferably, the blend is a co-processed blend, i.e. a blend obtained by co-processing citrus fibers and 1,3-β-D-glucan. As used herein, the term “mixing” refers to a blend wherein the 1,3-β-D-glucan is and the citrus fibers are mechanically blended together in a dry or substantially dry state. As used herein, the term “co-dried blend” refers to a blend wherein the 1,3-β-D-glucan is and the citrus fibers are mixed together in combination with water and/or a solvent and further dried to remove some, most, or all of the water and/or solvent. As used herein, the term “co-processed blend” refers to a blend wherein the 1,3-β-D-glucan is distributed between the citrus fibers and more in particular between the microfibrils forming the fibers, i.e. a majority of the 1,3-β-D-glucan is distributed between said microfibrils. Preferably, said blend is co-processed to distribute at least 10 wt % of the 1,3-β-D-glucan present in the blend between said microfibrils, more preferably at least 30 wt %, even more preferably at least 50 wt %, even more preferably at least 75 wt %, most preferably at least 90 wt %. The method of WO2017/019752 can be used to prepare a co-processed blend of sclaroglucan and citrus fiber. The co-processed blend is generally prepared by adding the 1,3-β-D-glucan in a dry, wet, liquid or solution form to a slurry of citrus fibers at any stages of the fiber's preparation process. It is preferably done during the steps of mechanical shearing. In general, citrus fibers undergo a high shear treatment, like high pressure homogenization or any shearing process known in the art. Adding the 1,3-β-D-glucan prior to this operation allows a distribution of the 1,3-β-D-glucan between the cellulose microfibrils. Alternatively, the 1,3-β-D-glucan can be added anywhere in the citrus fibers process. The blend can also be done in the other way, where dry, wet or water/solvent dispersed citrus fibers are added to the purification stages of the 1,3-β-D-glucan process i.e. any stages after pasteurization.

The concentration of citrus fibers in the blend may advantageously be between 25 wt % and 99 wt %. For example, the concentration of citrus fibers may be between 35 wt% to 95 wt %, preferably between 45 wt % and 90 wt %, most preferably between 55 wt % and 85 wt %. Preferably, these amounts are characteristic for the dry blend.

The concentration of 1,3-β-D-glucan in the blend may be between 0.1 wt % and 75 wt %. For example, the concentration of the 1,3-β-D-glucan may be between 1 wt % to 65 wt %, preferably between 5 wt % and 55 wt %, or between 10 wt % and 55 wt %, most preferably between most preferably between 15 wt % and 45 wt %. Preferably, these amounts are characteristic for the dry blend.

Alternatively, the dry blend may be characterized by the ratio of citrus fibers to 1,3-β-D-glucan. The ratio in the blend may be between 10:90 and 90:10, 20:80 and 80:20, 30:70 to 70:30, 60:40 and 40:60; or 55:45 and 45:55. A preferred ratio is 52:48.

The dry blend may optionally contain other ingredients binders, fillers, texturizers, emulsifiers, active ingredients, in any amount.

In one aspect, the invention provides an aqueous mixture comprising the dry blend disclosed herein. The term “aqueous mixture”, as used herein refers to a mixture created with any of the dry blends disclosed herein with a liquid phase that is greater than 50% water. The liquid phase may include other non-water solvents or materials such as alcohols or other organic solvents. Preferably the liquid phase is greater than 80% or 90% water. Preferably the liquid phase is 95% or 99+% water. In some embodiments the aqueous mixture or liquid phase contains less than 5% or 1% of any non-water solvent. In other embodiments the aqueous mixture or liquid phase contains water as solvent.

The aqueous mixture may advantageously have a viscosity of 3 to 300,000 Cps. For example, the viscosity of the aqueous solution may be between 200 to 27,000 Cps, 3,000 to 25,000 Cps, 4,500 to 20,000 Cps or between 5,000 to 15,000 Cps.

### Topical Formulation

The emulsion or aqueous mixture comprising the dry blend provided herein is useful in the manufacture of personal care products, such as topical formulations. The inventors unexpectedly found that formulations comprising a combination of citrus fibres and 1,3-β-D-glucan have numerous desirable characteristics as explained further below.

In one aspect, the present invention is a topical formulation comprising an emulsion or aqueous mixture comprising the dry blend as described herein. As used herein, the term “topical formulation” refers to a formulation that may be applied directly to a part of the body. The term “formulation” is used herein to denote compositions of various ingredients in various weight ranges, in accordance with the present invention.

The formulations manufactured with the emulsions or aqueous mixture comprising the dry blend described herein are suitable for use on hair, scalp, nails and skin, for delivering cosmetic or actives to the skin or hair for providing cleansing, conditioning, moisturizing, minimizing or treating skin imperfections, reducing skin oiliness, providing fragrances to the hair or skin and the like.

“Personal care” means and comprises any cosmetic, hygienic, toiletry and topical care products including, without limitation, leave-on products (i.e., products that are left on keratinous substrates after application); rinse-off products (i.e., products that are washed or rinsed from keratinous substrates during or within a few minutes of application); shampoos; hair curling and hair straightening products; hair style maintaining and hair conditioning products; lotions and creams for nails, hands, feet, face, scalp and/or body; hair dye; face and body makeup; nail care products; astringents; deodorants; antiperspirants; anti-acne; antiaging; depilatories; colognes and perfumes; skin protective creams and lotions (such as sunscreens); skin and body cleansers; skin conditioners; skin toners; skin firming compositions; skin tanning and lightening compositions; liquid soaps; bar soaps; bath products; shaving products; and oral hygiene products (such as toothpastes, oral suspensions, and mouth care products).

The texture of such personal care formulations is not limited and may be, without limitation, a liquid, gel, spray, emulsion (such as lotions and creams), shampoo, pomade, foam, tablet, stick (such as lip care products), makeup, suppositories, among others, any of which can be applied to the skin or hair or hale and which typically are designed to remain in contact therewith until removed, such as by rinsing with water or washing with shampoo or soap. Other forms could be gels that can be soft, stiff, or squeezable. Sprays can be non-pressurized aerosols delivered from manually pumped finger-actuated sprayers or can be pressurized aerosols such as mousse, spray, or foam forming formulation, where a chemical or gaseous propellant is used.

The topical formulation comprising the emulsion or aqueous mixture comprising the dry blend disclosed herein may be a shampoo. Advantageously, the shampoo may comprise co-processed citrus fibers and scleroglucan.

The topical formulation comprising an emulsion or aqueous mixture comprising the dry blend described herein may have a pH between from 3 and 8. Preferably, the pH of the topical formulation is between 4 and 7.

Formulations prepared using the emulsion or aqueous mixture comprising the dry blend disclosed herein have a white or off-white colour that is generally considered to be aesthetically appealing. In some cases, the formulations disclosed herein may be further processed to make a coloured end product. In such cases, the white or off-white colour is beneficial because it will show up the additional pigment without influencing the final colour.

The degree of whiteness can be determined using known methods, e.g. using a colour spectrometer and described in terms of the co-ordinates L*,a*, and b*. Preferably, the topical formulation has an L* value of from 66 to 83, an a* value from 0.3 to −1.1 and a b* value is from 3 to 10.

Furthermore, formulations prepared using the emulsion or aqueous mixture comprising the dry blend disclosed herein have a pleasant creamy and smooth texture. This texture feels pleasant to touch and apply. Furthermore, the consistency is such that good product pick-up may be achieved. Good product pick-up means that sufficient product (i.e. not too much, and not too little) can be collected on the user's finger.

Upon application, the formulations of the present invention have also been found to leave a fresh and pleasantly cooling skin feel.

### Process of Manufacturing Emulsions and Topical Formulations

The emulsion of the present invention may be formed by a process comprising the steps of:

a) providing a blend of citrus fibers and 1,3-β-D-glucan;
b) adding the blend to water and mixing to form an aqueous phase;
c) dispersing oil in the aqueous phase to obtain an emulsion.

The process may optionally further include a homogenization step before and/or after step c).

The 1,3-β-D-glucan may preferably be scleroglucan.

Advantageously, providing a blend may include providing a co-dryed or co-processed blend as described above.

The blend and water may be advantageously mixed between 5-20 minutes at 5000 rpm (e.g. using a Silverson homogenizer) at room temperature to form an aqueous phase. For example, the blend and water may be mixed for between 5-20 minutes, preferably for between 10-15 minutes, or more preferably for 10 minutes.

The aqueous phase and the oil phase may be heated before and/or during step c). The phases may be heated separately. In case an oil phase is used which is solid, semi-solid, or viscus at room temperature, the oil phase may be heated above its melting temperature in order to improve the process ability thereof. In case the oil phase should be heated, it is preferred that the aqueous phase is heated to at least the same temperature to which the oil phase is heated. Preferably, the heated phases are mixed whilst being stirred using a heated plate. Once the aqueous phase and the oil phase are heated, the oil may be dispersed in the aqueous phase.

Homogenization ideally takes place for at least 1 minute, more preferably at least 3 minutes, more preferably between 5-20 minutes at 5000 rpm (e.g. using a Silverson homogenizer). For example, the oil phase and the aqueous phase may be homogenized for between 5-20 minutes, preferably for between 10-15 minutes, or more preferably for 10 minutes.

In one aspect, the invention provides an emulsion comprising citrus fibers and scleroglucan obtainable by the above process.

An aqueous mixture comprising the dry blend can be prepared by utilizing steps a) and b) as described above.

A topical formulation may be obtained by carrying out steps a) and b), or steps a) to c), described above and through step d) which is subsequently adding at least one further ingredient to the emulsion and mixing to obtain a topical formulation.

The at least one further ingredient may be chosen from the group consisting of preservative, salt, vitamin, emulsifier, texturiser, nutrient, micronutrient, sugar, protein, polysaccharide, polyol, glucose, sucrose, glycerol, sorbitol, pH adjusters, emollients, dyes, pigments, skin actives, waxes or silicones.

Preferably, the preservative is Isaguard PEHG and/or salt (e.g. sodium chloride).

If the emulsion was heated above ambient temperature during its formation, it may be cooled before step d) and the further ingredient added once the emulsion has been cooled.

The emulsion and at least one further ingredient may be advantageously mixed for between 2 and 20 minutes at 5000 rpm at ambient or elevated temperature (e.g. using a Silverson homogenizer) to form the topical formulation. For example, the emulsion and at least one further ingredient may be mixed for between 2 and 10 minutes, or between 2 and 7 minutes, or preferably for between 2 and 5 minutes, or more preferably for 2 minutes. When more than one further ingredient is added, they may be added separately or together. The emulsion may be mixed after the addition of each or some of the further ingredient(s), or after all the further ingredients have been added.

Water may be lost throughout the process, for example due to evaporation. The process may therefore further include a step of checking for water loss and adding water back in to the topical formulation. The mixture may subsequently be mixed following the addition of water (e.g. at 5,000 rpm using a Silverson type Homogenizer” L5M-A Laboratory Mixer, Silverson for 2 minutes).

In one aspect, the invention may be a topical formulation comprising an emulsion and at least one further ingredient obtainable by the process disclosed above.

### EXAMPLES

### Materials and Measurement Methods

### Starting Materials

The citrus fibers used were CitriTex-ACF obtained from Cargill Incorporated. Alternatively, citrus fibers may be prepared using known protocols, for example as described in WO 2012/016190 and WO 2017/019752.

The scleroglucan used was Actigum™ CS 6 Scleroglucan, Actigum™ CS 11 QD, Actigum™ CS11 or Actigum CS QD I obtained from Cargill Incorporated. CS-6 is post-fermentation scleroglucan that has not been refined and contains 60-75% scleroglucan. CS-11 is a refined product; containing 85-90% scleroglucan.

### Emulsion/Topical Formulation Preparation

| | |
|---|---|
| Equipment | L5M-A Laboratory Mixer, Silverson |
| Details | Mixing speed 5000 rpm |
| | Mixing water phase: 10 minutes |
| | Mixing emulsion: 10 minutes |
| | Mixing after preservative: 2 minutes |
| | Mixing after salt addition: 5 minutes |
| Temperature | Room Temperature (22-24° C.) |
| of | However, with the shear of mixer the emulsion |
| ingredients | temperature increased to around 36° C. |
| and | 45° C. |
| emulsion | Oil phase and water phase were heated separately using |
| | a heating plate while being stirred to 45° C. Once |
| | they reached 45° C., the oil was added to the water |
| | phase and homogenized for 10 minutes. Cool the emulsion |
| | to below 40° C. before adding preservative. |
| Procedure | Add water to the beaker and start mixing with silverson |
| | at 5000 rpm (grid with small round holes) |
| | Add powder to the water and mix at 5000 rpm for 10 min. |
| | Add oil and mix with Silverson at 5000 rpm. Mix well |
| | (up/down) to ensurefull homogenization for 10 min. |
| | Add preservative and mix with Silverson at 5000 rpm for |
| | 2 min. |
| | Add NaCl if needed and mix with Silverson at 5000 rpm |
| | for 5 min. |
| | Check for water loss and add back water loss and mix |
| | with Silverson at 5000 rpm for 2 minutes. |

### Viscosity Measurements

| | |
|---|---|
| Equipment | Brookfield Viscometer DV-II + Pro, Ametek, USA |
| Temperature | All samples were measured at room temperature |
| | (22-24° C.). If samples were held at 45° C. |
| | in the Clima Temperatur Systeme, then samples were |
| | removed at 10:00 AM and allowed to cool for 6 hours |
| | until 16:00PM when the measurements were taken. |
| Details | 20 rpm |
| | 2-minute measurement |
| Measurement | Turn on the viscometer with the power in the back |
| | Select spindle number (1-7) Torque should be between |
| | 10 and 90% |
| | 20 rpm |
| | Inset the spindle, the sample should come to spindle |
| | mark |
| | After 2 minutes record the viscosity value, torque |
| | and spindle |

### pH Measurements

| Equipment | SevenMulti ™ pH meter, Mettler Toledo, USA | |
|---|---|---|
| Temperature | All samples were measured at room temperature | |
| | (22-24° C.). If samples were held at 45° C. | |
| | in the Clima Temperatur Systeme, then samples were | |
| | removed at 10:00 AM and allowed to cool for 6 hours | |
| | until 16:00PM when the measurements were taken. | |
| Calibration | Calibration was performed daily using two solutions | |
| | 7.00 and 401. | |
| Preparation | On/off | |
| | 1. | take out from electrode solution and rinse |
| | | with demi water |
| | 2. | clean electrode with wet tissue |
| | 3. | put the electrode in the sample submerged but |
| | | not far down. Use the holder to do this. |

| | |
|---|---|
| Measurement | Press the read button. When the measurement is finished |
| | record the pH value. |
| Clean | Clean the outside of the probe with a wet tissue to |
| | remove excess product |
| | Rinse the probe with demi water |
| | Put the probe back in the holding solution |
| | Turn off |

### Appearance/Stability Procedure

| Equipment | Clima Temperatur Systeme, CTS, Germany | |
|---|---|---|
| Temperature | All samples were measured at room temperature | |
| | (22-24° C.). If samples were held at 45° C. | |
| | in the Clima Temperatur Systeme, then samples | |
| | were removed at 10:00 AM and allowed to cool for | |
| | 6 hours until 16:00PM when the measurements were | |
| | taken. | |
| Details | Samples were considered stable if there were no | |
| Stability | oil droplets seen on the surface and no separation | |
| | observed throughout the sample (see FIG. 1). | |
| | Samples were considered to have moderate stability | |
| | if oil droplets were seen on the surface and/or | |
| | slight separation was observed in the emulsion. | |
| | Some oil crystals may also be seen on the surface | |
| | (see FIG. 7). | |
| | Samples were considered to have low stability if | |
| | a distinct line of oil separation was seen on the | |
| | surface or sides of the sample, and/or if pools | |
| | of oil and oil crystals were observed in the sample | |
| | (see FIG. 1). | |
| Definitions | Appearance of the samples was recorded. Terms were | |
| | given for | |
| Appearance | 1. | Color (see FIG. 2) |
| | | White |
| | | Off-white |
| | | Beige |
| | 2. | Reflection (see FIG. 3) |
| | | Shiny- high reflectance, shine |
| | | Matte - dull, low reflectance |
| | 3. | Texture |
| | | Smooth (see FIG. 4) - a uniform structure |
| | | where no balls are observed |
| | | Gritty/Grainy (see FIG. 4) - small balls or |
| | | pieces can be seen Jellied (see FIG. 6) - |
| | | flambee, short texture, sample pulls from |
| | | sides with spoon |
| | | Creamy (see FIG. 6) - does not pull from |
| | | the sides with spoon, thick and easy to |
| | | scoop with a spoon, not flambee. |
| | Compote - thick, chunky texture with large lumps | |
| | 4. | Pick-up (see FIG. 5) |

| | |
|---|---|
| | Low- product is difficult to pick up with a small |
| | spoon. When a finger is inserted only a little |
| | product comes out with the finger. |
| | Medium- moderate amount of product is able to be |
| | picked up with a small spoon. When a finger is |
| | inserted, it comes out with a moderate layer |
| | of product. |
| | High- a large amount of product is easy to be |
| | picked up with a small spoon. When a finger |
| | is inserted a large coating of product remains |
| | on the finger. |
| Rank | Numbers 1 to 5 were given to samples. 1 is the |
| | lowest and 5 is the highest. |

### Sensory Procedure

| | |
|---|---|
| Temperature | All samples were measured at room temperature |
| | (22-24° C.). Samples held at 45° C. were not |
| | tested. |
| Definitions | Pick-up- how much of the product is able to be picked up |
| | on the finger when the finger is touched to the surface |
| | of the product |
| | Fresh- a cooling feeling is felt on the skin after 2 |
| | circle rubs |
| | Oily/slippery- thick oil feel is felt on the skin after |
| | 5 circle rubs |
| | Absorption- the amount of time the sample requires to |
| | sink/penetrate into the skin |
| | Residue- any remaining product or substance left on the |
| | skin after absorption |
| | Watery- a feeling of water being applied to the skin |
| Rank | Number 1 to 5 were given to samples. 1 is the lowest and |
| | 5 is the highest. |
| Procedure | A small amount of sample was picked up with a finger. The |
| | sample was placed on the inside of the upper forearm. The |
| | sample was rubbed 5 times in a circular motion and then |
| | evaluated. |

### Colour Spectrometer Procedure

| Equipment | CM-5 Spectrophotometer, Konica Minolta, | |
|---|---|---|
| Temperature | All samples were measured at room temperature (22- | |
| | 24° C.). Samples held at 45° C. were not tested. | |
| Preparation | Turn machine on the right side | |
| | Menu | |
| | Measuring | reflections |
| | conditions | 3 mm (small) |
| | | SCE excluded |

| | |
|---|---|
| Calibration | Performed at the beginning of each measurement session |
| | Black calibration/Zero calibration |
| | White calibration |
| Details | Auto measurement 3 times |
| | Manual measurement 1 time |
| | Color (L*a*b*) deltaE00 |
| | Color index-none |
| | Observer- 10 degrees |
| | Illuminant 1-D65 |
| | Illuminant 2- none |
| Measurement | 1 sample was tested 3 times. This means that for |
| | each measurement 3 automatic measurements were taken |
| | and averaged. This average was taken 3 times and then |
| | averaged for a total of 9 measurements. CR-A504 Tube |
| | Cell 35 × 34mmØ was filled with around 10 g |
| | of sample (about 14 full). 3 automatic measurements |
| | were taken and averaged. The cell was emptied, cleaned |
| | with demi water, and fresh sample was placed in the |
| | cell to be measured again. The cell was then emptied and |
| | cleaned and then a third manual measurement was taken. |

### Water Holding Capacity (WHC)

| | | WHC (1 wt. % |
|---|---|---|
| | Dry | fiber in buffer) |
| Products | substance (%) | [g/g dry product] |
| Herbacel AQ-N | 91.69 | 24.70 |
| Citri-Tex ACF | 91.40 | 32.77 |
| *65% Citri-Tex ACF + 35% | 90.57 | >99 (no separation) |
| scleroglucan CS 6 | | |
| *65% Citri-Tex ACF + 35% | 91.88 | >99 (no separation) |
| scleroglucan CS 11 | | |
| *70% Citri-Tex ACF + 30% | 91.51 | 28.66 |
| Glycerol | | |

*refers to co-processed Citrus Fiber with additive

Water holding capacity is determined as follows.

1. A 1% wt fiber suspension was prepared by diluting a slurry of 2% wt fiber with Clark Lubs buffer (50 g of sample+50g of buffer);
2. A dispersion is achieved by means of a magnetic stirrer (30min) at speed 400 rpm;
3. Centrifuge tubes (with caps) were tarred (W₀);
4. The fiber suspension was quickly transferred into two centrifuge tubes and capped.
5. The tubes were balanced (W₁) so that they have exactly the same weights 2 by 2 (+/−0.1 g);
6. The tubes were centrifuged them at 3000 g for 10 minutes at room temperature;
7. The supernatant was removed by means of a pipette and weighed (W₂).
8. The packed material was weighed (W₃).
9. The Water Holding Capacity is calculated as follows: WHC=(W₃-W₀)/(W₁-W₀)*100 Water holding capacity for the samples prepared with the blend of present invention clearly demonstrate superior water holding capacity than the control formulations. This property is highly advantageous and desirable in personal care applications.

### Example 1

Emulsions and topical formulations were prepared with varying concentrations of co-processed citrus fibers and scleroglucan and tested in accordance with the methods and procedures described above. Co-processed citrus fibers Emulsions comprising citrus fibers or scleroglucan alone were also prepared as control samples. For comparative purposes, emulsions comprising citrus fibers and scleroglucan in the form of a blend were also tested, as well as emulsions comprising citrus fibers in combination with compounds other than scleroglucan. For example, CitriTex-ACF and glycerol, CitriTex-ACF and erythritol and CitriTex-ACF and Satiagel™ VPC 512 (Cargill Incorporated). In Table 1, all Emulsions were made with 2% weight citrus fibers and 20% weight Rapeseed oil and preserved. The results are shown in Table 1 below.

TABLE 1
All Emulsions made with 2% weight “as is” co-processed
citrus fibers and 20% weight Rapeseed oil, preserved.

| | | | | | Pick-up | Quick |
|---|---|---|---|---|---|---|
| | | | | | Low = 0 | Break/fast |
| | | | | | (low amount | to spread |
| | | | | | of product | on skin |
| Test | Concentrations | | Grainy = 0 | | on finger) | Slow = 0 |
| Description | in the blend | Color | Smooth = 5 | Texture | High =5 | Fast = 5 |
| COMPARATIVE - | 90% CitriTex- | Grey | 1 | Compote | 4 | 4 |
| Co-processed | ACF and 10% | Beige | | | | |
| Citrus Fibers | Glycerol | | | | | |
| and Glycerol | 80% CitriTex- | Grey | 1 | Compote | 4 | 4 |
| | ACF and 20% | Beige | | | | |
| | Glycerol | | | | | |
| | 70% CitriTex- | Grey | 1 | Compote | 4 | 4 |
| | ACF and 30% | Beige | | | | |
| | Glycerol | | | | | |
| Co-processed | 90% CitriTex- | Grey | 2 | Creamy - | 4 | 4 |
| Citrus Fibers | ACF and 10% | Beige | | medium | | |
| and Scleroglucan | scleroglucan | | | consistency | | |
| | 75% CitriTex- | Beige | 3 | Creamy - | 4 | 4 |
| | ACF and 25% | | | medium | | |
| | scleroglucan | | | consistency | | |
| | 50% CitriTex- | Off- | 4 | Creamy - | 4 | 4 |
| | ACF and 50% | white | | high | | |
| | scleroglucan | | | consistency | | |
| COMPARATIVE - | 90% CitriTex- | Grey | 1 | Compote | 4 | 4 |
| Co-processed | ACF and 10% | Beige | | | | |
| Citrus Fibers | erythritol | | | | | |
| and Erythritol | 75% CitriTex- | Grey | 1 | Compote | 3 | 4 |
| | ACF and 25% | Beige | | | | |
| | erythritol | | | | | |
| | 50% CitriTex- | Grey | 2 | Compote | 3 | 4 |
| | ACF and 50% | Beige | | | | |
| | erythritol | | | | | |
| COMPARATIVE - | 90% CitriTex- | Grey | 3 | Creamy | | |
| Co-processed | ACF and 10% | Beige | | fluid | | |
| Citrus Fibers | Satiagel VPC | | | | | |
| and Satiagel | 512 | | | | | |
| | 75% CitriTex- | Grey | 3 | Creamy | 4 | 3 |
| | ACF and 25% | Beige | | fluid | | |
| | Satiagel VPC | | | | | |
| | 512 | | | | | |
| | 50% CitriTex- | Grey | 3 | Creamy | 3 | 3 |
| | ACF and 50% | Beige | | fluid | | |
| | Satiagel VPC | | | | | |
| | 512 | | | | | |
| (Non- co- | 90% CitriTex- | Grey | 2 | Creamy - | 2 | 3 |
| processed) blend | ACF and 10% | Beige | | medium | | |
| of Citrus Fibers | scleroglucan | | | consistency | | |
| and Scleroglucan | 75% CitriTex- | Beige | 3 | Flamby- | 2 | 2 |
| | ACF and 25% | | | gelly | | |
| | scleroglucan | | | | | |
| | 50% CitriTex- | Off- | 4 | Flamby - | 1 | 2 |
| | ACF and 50% | beige | | gelly | | |
| | scleroglucan | | | | | |
| CONTROL - | Citri-Tex ACF | Grey | 0 | Compote | 4 | 4 |
| Citrus Fibers | | Beige | | | | |
| CONTROL- | CS11 | White | 5 | Flamby | 0 | 1 |
| Scleroglucan | Scleroglucan | | | very | | |
| | | | | gelly- | | |
| | | | | hard | | |
| | | | | texture | | |

| | | | | Peluchage - | |
|---|---|---|---|---|---|
| | | | | residue | |
| | | Speed of | Smooth | on skin | |
| | | penetration | after feel | after feel | |
| Test | Concentrations | Low = 0 | Low = 0 | No = 0 | |
| Description | in the blend | High = 5 | High = 5 | High = 5 | Stability |
| COMPARATIVE - | 90% CitriTex- | | | | |
| Co-processed | ACF and 10% | | | | |
| Citrus Fibers | Glycerol | | | | |
| and Glycerol | 80% CitriTex- | | | | Some unstability |
| | ACF and 20% | | | | signs visible. |
| | Glycerol | | | | Large + small oil |
| | | | | | droplets on the |
| | | | | | surface. Off-white |
| | | | | | color. Texture- |
| | | | | | structured. |
| | 70% CitriTex- | | | | |
| | ACF and 30% | | | | |
| | Glycerol | | | | |
| Co-processed | 90% CitriTex- | 2 | 3 | 2 | white ring around |
| Citrus Fibers | ACF and 10% | | | | edges, off-white |
| and Scleroglucan | scleroglucan | | | | color, texture- |
| | | | | | structured. |
| | 75% CitriTex- | 3 | 4 | 1 | Good stability, |
| | ACF and 25% | | | | Little Oil droplets |
| | scleroglucan | | | | on the surface, |
| | | | | | small amount of |
| | | | | | oil running around |
| | | | | | the edges, Off- |
| | | | | | white color. |
| | | | | | Texture- |
| | | | | | structured. |
| | 50% CitriTex- | 3 | 4 | 1 | Good stability, |
| | ACF and 50% | | | | very little oil in |
| | scleroglucan | | | | cracks, color-off- |
| | | | | | white, texture- |
| | | | | | very structured. |
| COMPARATIVE - | 90% CitriTex- | | | | |
| Co-processed | ACF and 10% | | | | |
| Citrus Fibers | erythritol | | | | |
| and Erythritol | 75% CitriTex- | | | | |
| | ACF and 25% | | | | |
| | erythritol | | | | |
| | 50% CitriTex- | | | | |
| | ACF and 50% | | | | |
| | erythritol | | | | |
| COMPARATIVE - | 90% CitriTex- | | | | |
| Co-processed | ACF and 10% | | | | |
| Citrus Fibers | Satiagel VPC | | | | |
| and Satiagel | 512 | | | | |
| | 75% CitriTex- | | | | Surface: |
| | ACF and 25% | | | | some small |
| | Satiagel VPC | | | | oil droplets. |
| | 512 | | | | Off-white color. |
| | | | | | Texture-runny. |
| | 50% CitriTex- | | | | |
| | ACF and 50% | | | | |
| | Satiagel VPC | | | | |
| | 512 | | | | |
| (Non- co- | 90% CitriTex- | 2 | 3 | 1 | |
| processed) blend | ACF and 10% | | | | |
| of Citrus Fibers | scleroglucan | | | | |
| and Scleroglucan | 75% CitriTex- | 2 | 2 | 2 | |
| | ACF and 25% | | | | |
| | scleroglucan | | | | |
| | 50% CitriTex- | 2 | 2 | 1 | |
| | ACF and 50% | | | | |
| | scleroglucan | | | | |
| CONTROL - | Citri-Tex ACF | 3 | 2 | 1 | Some unstability |
| Citrus Fibers | | | | | signs visible. |
| | | | | | Surface: med oil |
| | | | | | droplets, some oil |
| | | | | | running around |
| | | | | | edge. Off-white |
| | | | | | color. Texture- |
| | | | | | runny. |
| CONTROL- | CS11 | 2 | 2 | 1 | Stable |
| Scleroglucan | Scleroglucan | | | | |

The results show that an emulsion comprising co-processed citrus fibers and scleroglucan provide a topical formulation with a smooth, creamy texture which is fast to spread on skin. In particular, the results show that an emulsion comprising a ratio of 50:50 scleroglucan to citrus fibers has an off-white colour, and high stability.

In contrast, citrus fibers alone provide a grey beige formulation with a less desirable grainy texture and high degree of separation between the oil phase and aqueous phase. Whilst the results show that scleroglucan alone provides a formulation with a white colour and smooth texture, the formulation has a low pick-up.

Emulsions comprising citrus fibers co-processed with other compounds glycerol, erythritol and satiagel provided formulations with a degree of separation between the oil phase and aqueous phase and a less desirable grey beige colour.

### Example 2

Further emulsions and topical formulations were prepared with varying concentrations of co-processed citrus fibers and scleroglucan and tested in accordance with the methods and procedures described above after the following time intervals: 0 days (D0), 1 day (D1), 1 week (W1), 2 weeks (W2), 3 weeks (W3), 4 weeks (W4), 16 weeks (W16). Emulsions comprising citrus fibers, CS11 scleroglucan or CS6 scleroglucan alone were also prepared as control samples. For comparative purposes, emulsions comprising citrus fibers in combination with compounds other than scleroglucan. For example, CitriTex-ACF and erythritol and CitriTex-ACF and Satiagel™ VPC 512 (Cargill Incorporated).

TABLE 2
Viscosity

| Test | Concentrations | Viscosity (Cps) Room Temp | | | | | | Viscosity (Cps) 45° C. |
|---|---|---|---|---|---|---|---|---|
| Description | in the blend | D 0 | D 1 | W 1 | W 2 | W 4 | W 8 | W 1 |
| Co-processed | 65% CitriTex-ACF and | 9960 | 16700 | 17500 | 16800 | 15300 | 13900 | 16900 |
| Citrus Fibers | 35% CS11 scleroglucan | | | | | | | |
| and CS11 | 65% CitriTex-ACF and | 8240 | 13400 | 13700 | 13200 | 12000 | 11600 | 13800 |
| Scleroglucan | 35% CS11 scleroglucan | | | | | | | |
| | 65% CitriTex-ACF and | 8880 | 24400 | 28000 | 29300 | 26300 | 28000 | 13000 |
| | 35% CS11 scleroglucan | | | | | | | |
| | 65% CitriTex-ACF and | 7540 | 22100 | 26500 | 24100 | 25650 | 26000 | 10500 |
| | 35% CS11 scleroglucan | | | | | | | |
| | 65% CitriTex-ACF and | 4010 | 8700 | 6800 | 7560 | 6600 | 6820 | 6800 |
| | 35% CS11 scleroglucan | | | | | | | |
| | 65% CitriTex-ACF and | 4880 | 9520 | 8240 | 8780 | 14120 | 6780 | 7600 |
| | 35% CS11 scleroglucan | | | | | | | |
| | 65% CitriTex-ACF and | 6780 | 12500 | 10900 | 10900 | 10900 | 10000 | 10900 |
| | 35% CS11 scleroglucan | | | | | | | |
| | 65% CitriTex-ACF and | 1855 | 3880 | 3250 | 3690 | 3500 | 3700 | 3060 |
| | 35% CS11 scleroglucan | | | | | | | |
| | 65% CitriTex-ACF and | 2470 | 5130 | 4580 | 4260 | 4360 | 4430 | 4050 |
| | 35% CS11 scleroglucan | | | | | | | |
| | 65% CitriTex-ACF and | 3410 | 6500 | 6040 | 5620 | 6100 | 5500 | 5600 |
| | 35% CS11 scleroglucan | | | | | | | |
| | 65% CitriTex-ACF and | 652 | 940 | 1005 | 1015 | 990 | 1020 | 915 |
| | 35% CS11 scleroglucan | | | | | | | |
| | 65% CitriTex-ACF and | 832 | 1210 | 1300 | 1325 | 1280 | 1300 | 1170 |
| | 35% CS11 scleroglucan | | | | | | | |
| | 65% CitriTex-ACF and | 1044 | 1600 | 1720 | 1680 | 1710 | 1655 | 1570 |
| | 35% CS11 scleroglucan | | | | | | | |
| | 65% CitriTex-ACF and | 6800 | 11500 | 12600 | 11800 | 11500 | 10500 | 11700 |
| | 35% CS11 scleroglucan | | | | | | | |
| | 65% CitriTex-ACF and | 11200 | 18000 | 18300 | 17400 | 18350 | 17400 | 19000 |
| | 35% CS11 scleroglucan | | | | | | | |
| | 75% CitriTex-ACF and | 3580 | 5960 | 6760 | 6440 | 6600 | 6440 | 4960 |
| | 25% CS11 scleroglucan | | | | | | | |
| | 75% CitriTex-ACF and | 5100 | 14550 | 12650 | 14700 | 14120 | 15000 | 7200 |
| | 25% CS11 scleroglucan | | | | | | | |
| | 75% CitriTex-ACF and | 4500 | 13440 | 10900 | 11280 | 10900 | 11500 | 6220 |
| | 25% CS11 scleroglucan | | | | | | | |
| | 75% CitriTex-ACF and | 6180 | 11120 | 12500 | 12400 | 12180 | 11140 | 7780 |
| | 25% CS11 scleroglucan | | | | | | | |
| | 75% CitriTex-ACF and | 5080 | 7740 | 8060 | 8700 | 8060 | 7340 | 6220 |
| | 25% CS11 scleroglucan | | | | | | | |
| | 75% CitriTex-ACF and | 7900 | 28500 | 27850 | 26900 | 27700 | 28300 | 7140 |
| | 25% CS11 scleroglucan | | | | | | | |
| Co-processed | 65% CitriTex-ACF and | 7380 | 8750 | 8140 | 7900 | 8200 | 7440 | 8380 |
| Citrus Fibers | 35% CS6 scleroglucan | | | | | | | |
| and CS6 | 65% CitriTex-ACF and | 6600 | 7400 | 7220 | 6620 | 6400 | 6200 | 7300 |
| Scleroglucan | 35% CS6 scleroglucan | | | | | | | |
| | 65% CitriTex-ACF and | 7440 | 14120 | 12800 | 16200 | 13700 | 11200 | 7600 |
| | 35% CS6 scleroglucan | | | | | | | |
| | 65% CitriTex-ACF and | 6500 | 14540 | 15700 | 11700 | 14500 | 14500 | 6850 |
| | 35% CS6 scleroglucan | | | | | | | |
| | 65% CitriTex-ACF and | 1970 | 2300 | 2170 | 2200 | 2240 | 2160 | 2350 |
| | 35% CS6 scleroglucan | | | | | | | |
| | 65% CitriTex-ACF and | 444 | 520 | 490 | 505 | 530 | 550 | 555 |
| | 35% CS6 scleroglucan | | | | | | | |
| COMPARTITIVE - | 90% CitriTex-ACF and | 3320 | 16260 | 14800 | 12300 | 13240 | 11600 | 5420 |
| Co-processed | 10% Satiagel VPC 512 | | | | | | | |
| Citrus Fibers | 75% CitriTex-ACF and | 1765 | 7340 | 7900 | 7380 | 7420 | 7960 | 4100 |
| and Satiagel | 25% Satiagel VPC 512 | | | | | | | |
| | 75% CitriTex-ACF and | 3800 | 18200 | 20950 | 22700 | 23200 | 20900 | 9000 |
| | 25% Satiagel VPC 512 | | | | | | | |
| | 75% CitriTex-ACF and | 2850 | 7070 | 7680 | 7400 | 7140 | 8200 | 5440 |
| | 25% Satiagel VPC 512 | | | | | | | |
| | 75% CitriTex-ACF and | 3510 | 12180 | 13050 | 12200 | 12360 | 14000 | 5900 |
| | 25% Satiagel VPC 512 | | | | | | | |
| | 75% CitriTex-ACF and | 6140 | 19350 | 26050 | 25500 | 25000 | 27150 | 2700 |
| | 25% Satiagel VPC 512 | | | | | | | |
| | 75% CitriTex-ACF and | 2390 | 9580 | 13100 | 12440 | 12800 | 14000 | 5020 |
| | 25% Satiagel VPC 512 | | | | | | | |
| | 65% CitriTex-ACF and | 3310 | 13100 | 15020 | 14520 | 15000 | 14980 | 5920 |
| | 35% Satiagel VPC 512 | | | | | | | |
| COMPARATIVE - | 65% CitriTex-ACF and | 6520 | 12000 | 11000 | 10600 | 10800 | 10300 | 10280 |
| Co-processed | 25% CS11 and 10% | | | | | | | |
| Citrus Fibers, | erytritol | | | | | | | |
| Scleroglucan, | | | | | | | | |
| and Erythritol | | | | | | | | |
| Control - | Citri-Tex ACF | 6000 | 20000 | 27000 | 24200 | | | 13000 |
| Citrus Fibers | | | | | | | | |
| Control - | CS11 Scleroglucan | 13000 | 30500 | 33800 | 32200 | 31200 | 28000 | 22200 |
| Scleroglucan | CS11 Scleroglucan | 10080 | 22800 | 23800 | 25600 | 25000 | 22300 | 19900 |
| Control - | CS6 Scleroglucan | 9260 | 11450 | 10700 | 10760 | 10500 | 10400 | 11600 |
| Scleroglucan | CS6 Scleroglucan | 8220 | 11800 | 13500 | 13100 | 11700 | 11020 | 10700 |

| Test | Concentrations | Viscosity (Cps) 45° C. | | | CPF | Rapeseed | Coconut |
|---|---|---|---|---|---|---|---|
| Description | in the blend | W 2 | W 4 | W 8 | % | oil % | oil % |
| Co-processed | 65% CitriTex-ACF and | 17500 | 16000 | 14400 | 2 | 20 | |
| Citrus Fibers | 35% CS11 scleroglucan | | | | | | |
| and CS11 | 65% CitriTex-ACF and | 12900 | 11700 | 11800 | 2 | 20 | |
| Scleroglucan | 35% CS11 scleroglucan | | | | | | |
| | 65% CitriTex-ACF and | 12300 | 9200 | 15600 | 2 | | 20 |
| | 35% CS11 scleroglucan | | | | | | |
| | 65% CitriTex-ACF and | 10400 | 7680 | 12400 | 2 | | 20 |
| | 35% CS11 scleroglucan | | | | | | |
| | 65% CitriTex-ACF and | 6500 | 6000 | 6100 | 1.5 | 10 | |
| | 35% CS11 scleroglucan | | | | | | |
| | 65% CitriTex-ACF and | 7800 | 7300 | 6520 | 1.5 | 20 | |
| | 35% CS11 scleroglucan | | | | | | |
| | 65% CitriTex-ACF and | 10800 | 10200 | 9900 | 1.5 | 30 | |
| | 35% CS11 scleroglucan | | | | | | |
| | 65% CitriTex-ACF and | 2950 | 2800 | 2800 | 1 | 10 | |
| | 35% CS11 scleroglucan | | | | | | |
| | 65% CitriTex-ACF and | 3760 | 3800 | 3590 | 1 | 20 | |
| | 35% CS11 scleroglucan | | | | | | |
| | 65% CitriTex-ACF and | 5500 | 5300 | 5000 | 1 | 30 | |
| | 35% CS11 scleroglucan | | | | | | |
| | 65% CitriTex-ACF and | 900 | 905 | 900 | 0.5 | 10 | |
| | 35% CS11 scleroglucan | | | | | | |
| | 65% CitriTex-ACF and | 1145 | 1190 | 1175 | 0.5 | 20 | |
| | 35% CS11 scleroglucan | | | | | | |
| | 65% CitriTex-ACF and | 1540 | 1490 | 1505 | 0.5 | 30 | |
| | 35% CS11 scleroglucan | | | | | | |
| | 65% CitriTex-ACF and | 10700 | 10400 | 9560 | 2 | 10 | |
| | 35% CS11 scleroglucan | | | | | | |
| | 65% CitriTex-ACF and | 18700 | 18000 | 16600 | 2 | 30 | |
| | 35% CS11 scleroglucan | | | | | | |
| | 75% CitriTex-ACF and | 4630 | 5060 | 4610 | 2 | 10 | |
| | 25% CS11 scleroglucan | | | | | | |
| | 75% CitriTex-ACF and | 5370 | | | 2 | | 30 |
| | 25% CS11 scleroglucan | | | | | | |
| | 75% CitriTex-ACF and | 5700 | 5680 | 6800 | 2 | | 20 |
| | 25% CS11 scleroglucan | | | | | | |
| | 75% CitriTex-ACF and | 6820 | 6120 | 6760 | 2 | | 20 |
| | 25% CS11 scleroglucan | | | | | | |
| | 75% CitriTex-ACF and | 6200 | 5880 | 5660 | 2 | | 10 |
| | 25% CS11 scleroglucan | | | | | | |
| | 75% CitriTex-ACF and | | | | 2 | | 30 |
| | 25% CS11 scleroglucan | | | | | | |
| Co-processed | 65% CitriTex-ACF and | 8260 | 7620 | 7200 | 2 | 20 | |
| Citrus Fibers | 35% CS6 scleroglucan | | | | | | |
| and CS6 | 65% CitriTex-ACF and | 6980 | 6500 | 5700 | 2 | 20 | |
| Scleroglucan | 35% CS6 scleroglucan | | | | | | |
| | 65% CitriTex-ACF and | 8120 | 7000 | 7300 | 2 | | 20 |
| | 35% CS6 scleroglucan | | | | | | |
| | 65% CitriTex-ACF and | 6860 | 6500 | 5700 | 2 | 20 | 20 |
| | 35% CS6 scleroglucan | | | | | | |
| | 65% CitriTex-ACF and | 2500 | 2415 | 2455 | 1 | 20 | |
| | 35% CS6 scleroglucan | | | | | | |
| | 65% CitriTex-ACF and | 570 | 600 | 650 | 0.5 | 10 | |
| | 35% CS6 scleroglucan | | | | | | |
| COMPARTITIVE - | 90% CitriTex-ACF and | 4700 | 5310 | 4380 | 2 | | 20 |
| Co-processed | 10% Satiagel VPC 512 | | | | | | |
| Citrus Fibers | 75% CitriTex-ACF and | 3760 | 2890 | 2700 | 2 | | 10 |
| and Satiagel | 25% Satiagel VPC 512 | | | | | | |
| | 75% CitriTex-ACF and | 5080 | | | 2 | | 30 |
| | 25% Satiagel VPC 512 | | | | | | |
| | 75% CitriTex-ACF and | 4820 | 4440 | 3920 | 2 | | 10 |
| | 25% Satiagel VPC 512 | | | | | | |
| | 75% CitriTex-ACF and | 5000 | 6960 | 5040 | 2 | | 20 |
| | 25% Satiagel VPC 512 | | | | | | |
| | 75% CitriTex-ACF and | | | | 2 | | 30 |
| | 25% Satiagel VPC 512 | | | | | | |
| | 75% CitriTex-ACF and | 6180 | 3100 | 3600 | 2 | | 20 |
| | 25% Satiagel VPC 512 | | | | | | |
| | 65% CitriTex-ACF and | 6300 | 5700 | | 2 | | 20 |
| | 35% Satiagel VPC 512 | | | | | | |
| COMPARATIVE - | 65% CitriTex-ACF and | 9860 | 9080 | 8800 | 2 | 20 | |
| Co-processed | 25% CS11 and 10% | | | | | | |
| Citrus Fibers, | erytritol | | | | | | |
| Scleroglucan, | | | | | | | |
| and Erythritol | | | | | | | |
| Control - | Citri-Tex ACF | 11900 | | | 2 | 20 | |
| Citrus Fibers | | | | | | | |
| Control - | CS11 Scleroglucan | 13000 | 10100 | 13900 | 2 | 20 | |
| Scleroglucan | CS11 Scleroglucan | 17500 | 21500 | 16600 | 2 | | 10 |
| Control - | CS6 Scleroglucan | 11760 | 11300 | 10800 | 2 | 20 | |
| Scleroglucan | CS6 Scleroglucan | 9800 | 9880 | 9800 | 2 | | 20 |

TABLE 3
pH

| | | | | | | | | | | | COMPOSITION | | |
|---|---|---|---|---|---|---|---|---|---|---|---|---|---|
| Test | Concentrations | pH Room Temp | | | | | pH 45° C. | | | | CPF | Rapeseed | Coconut |
| Description | in the blend | D 1 | W 1 | W 2 | W 4 | W 8 | W 1 | W 2 | W 4 | W 8 | % | oil % | oil % |
| Co-processed | 65% CitriTex- | 4.55 | 4.68 | 4.51 | 4.55 | 4.44 | 4.55 | 4.43 | 4.45 | 4.32 | 2 | 20 | |
| Citrus Fibers | ACF and 35% | | | | | | | | | | | | |
| and CS11 | CS11 | | | | | | | | | | | | |
| Scleroglucan | scleroglucan | | | | | | | | | | | | |
| | 65% CitriTex- | 4.38 | 4.91 | 4.39 | 4.39 | 4.35 | 4.68 | 4.33 | 4.33 | 4.26 | 2 | 20 | |
| | ACF and 35% | | | | | | | | | | | | |
| | CS11 | | | | | | | | | | | | |
| | scleroglucan | | | | | | | | | | | | |
| | 65% CitriTex- | 4.64 | 4.67 | 4.48 | 4.51 | 4.36 | 4.61 | 4.44 | 4.46 | 4.41 | 2 | | 20 |
| | ACF and 35% | | | | | | | | | | | | |
| | CS11 | | | | | | | | | | | | |
| | scleroglucan | | | | | | | | | | | | |
| | 65% CitriTex- | 4.36 | 4.39 | 4.36 | 4.36 | 4.32 | 4.34 | 4.33 | 4.35 | 4.28 | 2 | | 20 |
| | ACF and 35% | | | | | | | | | | | | |
| | CS11 | | | | | | | | | | | | |
| | scleroglucan | | | | | | | | | | | | |
| | 65% CitriTex- | 4.59 | 4.73 | 4.71 | 4.67 | 4.57 | 4.59 | 4.54 | 4.54 | 4.4 | 1.5 | 10 | |
| | ACF and 35% | | | | | | | | | | | | |
| | CS11 | | | | | | | | | | | | |
| | scleroglucan | | | | | | | | | | | | |
| | 65% CitriTex- | 4.51 | 4.60 | 4.66 | 4.62 | 4.54 | 4.52 | 4.55 | 4.49 | 4.4 | 1.5 | 20 | |
| | ACF and 35% | | | | | | | | | | | | |
| | CS11 | | | | | | | | | | | | |
| | scleroglucan | | | | | | | | | | | | |
| | 65% CitriTex- | 4.57 | 4.54 | 4.51 | 4.54 | 4.44 | 4.47 | 4.45 | 4.43 | 4.36 | 1.5 | 30 | |
| | ACF and 35% | | | | | | | | | | | | |
| | CS11 | | | | | | | | | | | | |
| | scleroglucan | | | | | | | | | | | | |
| | 65% CitriTex- | 4.77 | 4.72 | 4.44 | 4.71 | 4.75 | 4.69 | 4.55 | 4.58 | 4.5 | 1 | 10 | |
| | ACF and 35% | | | | | | | | | | | | |
| | CS11 | | | | | | | | | | | | |
| | scleroglucan | | | | | | | | | | | | |
| | 65% CitriTex- | 4.65 | 4.62 | 4.71 | 4.64 | 4.6 | 4.57 | 4.56 | 4.53 | 4.42 | 1 | 20 | |
| | ACF and 35% | | | | | | | | | | | | |
| | CS11 | | | | | | | | | | | | |
| | scleroglucan | | | | | | | | | | | | |
| | 65% CitriTex- | 4.64 | 4.66 | 4.56 | 4.55 | 4.54 | 4.50 | 4.51 | 4.44 | 4.34 | 1 | 30 | |
| | ACF and 35% | | | | | | | | | | | | |
| | CS11 | | | | | | | | | | | | |
| | scleroglucan | | | | | | | | | | | | |
| | 65% CitriTex- | 4.80 | 4.90 | 4.96 | 4.99 | 4.56 | 4.81 | 4.74 | 4.71 | 4.59 | 0.5 | 10 | |
| | ACF and 35% | | | | | | | | | | | | |
| | CS11 | | | | | | | | | | | | |
| | scleroglucan | | | | | | | | | | | | |
| | 65% CitriTex- | 4.69 | 4.79 | 4.73 | 4.72 | 4.73 | 4.72 | 4.65 | 4.65 | 4.5 | 0.5 | 20 | |
| | ACF and 35% | | | | | | | | | | | | |
| | CS11 | | | | | | | | | | | | |
| | scleroglucan | | | | | | | | | | | | |
| | 65% CitriTex- | 4.72 | 4.79 | 4.67 | 4.64 | 4.65 | 4.60 | 4.55 | 4.55 | 4.39 | 0.5 | 30 | |
| | ACF and 35% | | | | | | | | | | | | |
| | CS11 | | | | | | | | | | | | |
| | scleroglucan | | | | | | | | | | | | |
| | 65% CitriTex- | 4.58 | 4.58 | 4.59 | | | 4.47 | 4.50 | | | 2 | 10 | |
| | ACF and 35% | | | | | | | | | | | | |
| | CS11 | | | | | | | | | | | | |
| | scleroglucan | | | | | | | | | | | | |
| | 65% CitriTex- | 4.59 | 4.45 | 4.50 | | | 4.40 | 4.41 | | | 2 | 30 | |
| | ACF and 35% | | | | | | | | | | | | |
| | CS11 | | | | | | | | | | | | |
| | scleroglucan | | | | | | | | | | | | |
| | 75% CitriTex- | 4.11 | 4.06 | 4.07 | 4.02 | 4.04 | 4.06 | 3.98 | 3.97 | 3.99 | 2 | 10 | |
| | ACF and 25% | | | | | | | | | | | | |
| | CS11 | | | | | | | | | | | | |
| | scleroglucan | | | | | | | | | | | | |
| | 75% CitriTex- | | 3.95 | 4.00 | 3.95 | 3.95 | 4.03 | 3.91 | | | 2 | | 30 |
| | ACF and 25% | | | | | | | | | | | | |
| | CS11 | | | | | | | | | | | | |
| | scleroglucan | | | | | | | | | | | | |
| | 75% CitriTex- | 4.04 | 4.05 | 4.02 | 4.04 | 4.03 | 4.00 | 3.98 | 3.95 | 3.93 | 2 | | 20 |
| | ACF and 25% | | | | | | | | | | | | |
| | CS11 | | | | | | | | | | | | |
| | scleroglucan | | | | | | | | | | | | |
| | 75% CitriTex- | 4.48 | 4.31 | 4.32 | 4.34 | 4.32 | 4.31 | 4.28 | 4.30 | 4;23 | 2 | | 20 |
| | ACF and 25% | | | | | | | | | | | | |
| | CS11 | | | | | | | | | | | | |
| | scleroglucan | | | | | | | | | | | | |
| | 75% CitriTex- | 4.28 | 4.37 | 4.37 | 4.39 | 4.36 | 4.34 | 4.31 | 4.32 | 4.29 | 2 | | 10 |
| | ACF and 25% | | | | | | | | | | | | |
| | CS11 | | | | | | | | | | | | |
| | scleroglucan | | | | | | | | | | | | |
| | 75% CitriTex- | 4.39 | 4.32 | 4.22 | 4.29 | 4.32 | 4.30 | 4.3 | 4.28 | 4.29 | 2 | | 30 |
| | ACF and 25% | | | | | | | | | | | | |
| | CS11 | | | | | | | | | | | | |
| | scleroglucan | | | | | | | | | | | | |
| Co-processed | 65% CitriTex- | 4.68 | 4.78 | 4.73 | 4.59 | 4.65 | 4.58 | 4.58 | 4.49 | 4.41 | 2 | 20 | |
| Citrus Fibers | ACF and 35% | | | | | | | | | | | | |
| and CS6 | CS6 | | | | | | | | | | | | |
| Scleroglucan | scleroglucan | | | | | | | | | | | | |
| | 65% CitriTex- | 4.54 | 4.48 | 4.53 | 4.46 | 4.44 | 4.45 | 4.42 | 4.38 | 4.29 | 2 | 20 | |
| | ACF and 35% | | | | | | | | | | | | |
| | CS6 | | | | | | | | | | | | |
| | scleroglucan | | | | | | | | | | | | |
| | 65% CitriTex- | 4.52 | 4.64 | 3.67 | 4.53 | 4.51 | 4.61 | 4.44 | 4.46 | 4.41 | 2 | | 20 |
| | ACF and 35% | | | | | | | | | | | | |
| | CS6 | | | | | | | | | | | | |
| | scleroglucan | | | | | | | | | | | | |
| | 65% CitriTex- | 4.46 | 4.36 | 4.39 | 4.36 | 4.36 | 4.34 | 4.33 | 4.35 | 4.28 | 2 | 20 | 20 |
| | ACF and 35% | | | | | | | | | | | | |
| | CS6 | | | | | | | | | | | | |
| | scleroglucan | | | | | | | | | | | | |
| | 65% CitriTex- | 4.88 | 4.84 | 4.78 | 4.72 | 4.71 | 4.68 | 4.70 | 4.64 | 4.53 | 1 | 20 | |
| | ACF and 35% | | | | | | | | | | | | |
| | CS6 | | | | | | | | | | | | |
| | scleroglucan | | | | | | | | | | | | |
| | 65% CitriTex- | 5.04 | 5.08 | 4.91 | 4.86 | 4.82 | 4.92 | 4.83 | 4.85 | 4.68 | 0.5 | 10 | |
| | ACF and 35% | | | | | | | | | | | | |
| | CS6 | | | | | | | | | | | | |
| | scleroglucan | | | | | | | | | | | | |
| COMPARATIVE - | 90% CitriTex- | 3.67 | 3.66 | 3.66 | 3.67 | 3.70 | 3.64 | 3.67 | 3.60 | 3.55 | 2 | | 20 |
| Co-processed | ACF and 10% | | | | | | | | | | | | |
| Citrus Fibers | Satiagel VPC | | | | | | | | | | | | |
| and Satiagel | 512 | | | | | | | | | | | | |
| | 75% CitriTex- | 3.89 | 3.74 | 3.82 | 3.82 | 3.82 | 3.81 | 3.81 | 3.76 | 3.66 | 2 | | 10 |
| | ACF and 25% | | | | | | | | | | | | |
| | Satiagel VPC | | | | | | | | | | | | |
| | 512 | | | | | | | | | | | | |
| | 75% CitriTex- | 3.78 | 3.94 | 3.82 | 3.70 | 3.82 | 3.81 | 3.74 | | | 2 | | 30 |
| | ACF and 25% | | | | | | | | | | | | |
| | Satiagel VPC | | | | | | | | | | | | |
| | 512 | | | | | | | | | | | | |
| | 75% CitriTex- | 4.14 | 4.08 | 4.06 | 4.08 | 4.05 | 4.04 | 4.07 | 4.01 | 4.04 | 2 | | 10 |
| | ACF and 25% | | | | | | | | | | | | |
| | Satiagel VPC | | | | | | | | | | | | |
| | 512 | | | | | | | | | | | | |
| | 75% CitriTex- | 4.03 | 3.98 | 4.03 | 4.05 | 4.06 | 3.99 | 3.95 | 3.98 | 3.95 | 2 | | 20 |
| | ACF and 25% | | | | | | | | | | | | |
| | Satiagel VPC | | | | | | | | | | | | |
| | 512 | | | | | | | | | | | | |
| | 75% CitriTex- | 4.16 | 3.93 | 3.94 | 4.00 | 3.94 | 3.94 | | | | 2 | | 30 |
| | ACF and 25% | | | | | | | | | | | | |
| | Satiagel VPC | | | | | | | | | | | | |
| | 512 | | | | | | | | | | | | |
| | 75% CitriTex- | 3.86 | 3.77 | 3.78 | 3.74 | 3.62 | 3.73 | 3.73 | 3.67 | 3.55 | 2 | | 20 |
| | ACF and 25% | | | | | | | | | | | | |
| | Satiagel VPC | | | | | | | | | | | | |
| | 512 | | | | | | | | | | | | |
| | 65% CitriTex- | 4.21 | 4.17 | 4.17 | 4.2 | 4.15 | 4.11 | 4.11 | 4.09 | 3.97 | 2 | | 20 |
| | ACF and 35% | | | | | | | | | | | | |
| | Satiagel VPC | | | | | | | | | | | | |
| | 512 | | | | | | | | | | | | |
| COMPARATIVE - | 65% CitriTex- | 4.64 | 4.45 | 4.46 | 4.46 | 4.44 | 4.41 | 4.45 | 4.38 | 4.26 | 2 | 20 | |
| Co-processed | ACF and 25% | | | | | | | | | | | | |
| Citrus Fibers, | CS11 and 10% | | | | | | | | | | | | |
| Scleroglucan, | erytritol | | | | | | | | | | | | |
| and Erythritol | | | | | | | | | | | | | |
| Control - Citrus | Citri-Tex ACF | 4.27 | 4.24 | 4.24 | 4.24 | | 4.21 | 4.21 | | | 2 | 20 | |
| Fibers | | | | | | | | | | | | | |
| Control - | CS11 | 6.35 | 6.32 | 6.43 | 6.43 | 6.37 | 6.39 | 6.40 | 6.41 | | 2 | 20 | |
| Scleroglucan | Scleroglucan | | | | | | | | | | | | |
| | CS11 | 6.58 | 6.38 | 6.61 | 6.53 | 6.52 | 6.57 | 6.59 | 6.71 | | 2 | | 10 |
| | Scleroglucan | | | | | | | | | | | | |
| Control - | CS6 | 6.81 | 6.71 | 6.73 | | | 6.46 | | | | 2 | 20 | |
| Scleroglucan | Scleroglucan | | | | | | | | | | | | |
| | CS6 | 6.79 | 6.61 | 6.82 | 6.60 | 6.54 | 6.41 | 6.30 | 6.08 | | 2 | | 20 |
| | Scleroglucan | | | | | | | | | | | | |

The results in Table 3 show that an emulsion comprising co-processed citrus fibers and scleroglucan provide a topical formulation with a pH of 4-5. In particular, the results demonstrate that an emulsion comprising co-processed citrus fibers and scleroglucan provides a topical formulation that maintains a similar pH over the 4-week period.

Emulsions comprising citrus fibers co-processed with satiagel provided formulations with a more acidic pH. In contrast, scleroglucan alone provides a formulation with a more alkaline pH.

TABLE 4
Sensory

| | | Sensory at Room Temp | | | | | | |
|---|---|---|---|---|---|---|---|---|
| Test | Concentrations | | | | W2 | | | |
| Description | in the blend | D 0 | D 1 | W 1 | Pick-up | Fresh | Absorption | Oily |
| Co-processed | 65% CitriTex-ACF | Soft, fresh, | med pick-up, | fresh, oil | 2 | 5 | 5 | 1 |
| Citrus Fibers | and 35% CS11 | slightly oily | watery, fresh | absorbs nice | | | | |
| and CS11 | scleroglucan | on hand | no residue | | | | | |
| Scleroglucan | 65% CitriTex-ACF | oily, but soft | watery | fresh, cool, a | 3 | 4 | 3 | 3 |
| | and 35% CS11 | and no residue | application, | little oily | | | | |
| | scleroglucan | | but oily after | | | | | |
| | | | feel | | | | | |
| | 65% CitriTex-ACF | high pick-up, | nice lotion | fresh at first, | 3 | 2 | 2 | 4 |
| | and 35% CS11 | oily | feel, but oily, | but then oily | | | | |
| | scleroglucan | | no residue | | | | | |
| | 65% CitriTex-ACF | | light, fresh, | water, light, | 2 | 3 | 3 | 2 |
| | and 35% CS11 | | not oily | smooth | | | | |
| | scleroglucan | | | | | | | |
| | 65% CitriTex-ACF | thin and | more oily | smooth, fresh, | 3 | 2 | 2 | 3 |
| | and 35% CS11 | slightly oily | than 10%, | heavier than | | | | |
| | scleroglucan | | low pick-up | 10% | | | | |
| | 65% CitriTex-ACF | Too oily | too oily and | beginning is | 4 | 2 | 2 | 4 |
| | and 35% CS11 | | slippery | watery, but | | | | |
| | scleroglucan | | | then too oily | | | | |
| | 65% CitriTex-ACF | nice, fresh, | smooth, | watery, fresh, | 1 | 4 | 4 | 2 |
| | and 35% CS11 | watery, no | watery, skin | hydrating | | | | |
| | scleroglucan | pick-up | is soft | | | | | |
| | 65% CitriTex-ACF | oily | light and | smooth and | 1 | 2 | 2 | 4 |
| | and 35% CS11 | | watery, very | fresh, oily | | | | |
| | scleroglucan | | oily after feel | after feel | | | | |
| | 65% CitriTex-ACF | oily | too heavy | too heavy and | 2 | 1 | 1 | 5 |
| | and 35% CS11 | | and oily on | oily | | | | |
| | scleroglucan | | skin | | | | | |
| | 65% CitriTex-ACF | | fresh, | sensory nice, | 3 | 2 | 3 | 2 |
| | and 35% CS11 | | smooth, | smooth, fresh | | | | |
| | scleroglucan | | thick, but | | | | | |
| | | | absorbs | | | | | |
| | | | quick | | | | | |
| | 65% CitriTex-ACF | watery, but | heavy and | heavier and | 5 | 3 | 2 | 4 |
| | and 35% CS11 | then very oily | oily | slightly oily | | | | |
| | scleroglucan | after feel | | after feel | | | | |
| Co-processed | 65% CitriTex-ACF | oily and | thin and | fresh smooth, | 3 | 2 | 3 | 3 |
| Citrus Fibers | and 35% CS6 | heavy | fresh, but | but oily and | | | | |
| and CS6 | scleroglucan | | then slippery | slippery | | | | |
| Scleroglucan | | | and oily | | | | | |
| | 65% CitriTex-ACF | thin and oily, | thin and oily, | thin and oily, | 1 | 4 | 4 | 1 |
| | and 35% CS6 | too much oil | too much oil | too much oil | | | | |
| | scleroglucan | | | | | | | |

TABLE 5
Colour Spectrometer

| | | Colour Spectrometer | | | | | | | | | | | |
|---|---|---|---|---|---|---|---|---|---|---|---|---|---|
| Test | Concentrations | L* | | | | a* | | | | b* | | | |
| Description | in the blend | L* | L* | L* | Average | a* | a* | a* | Average | b* | b* | b* | Average |
| Co-processed | 65% CitriTex- | 78.11 | 78.02 | 78.08 | 78.07 | −0.14 | -0.12 | −0.15 | −0.14 | 7.45 | 7.58 | 7.32 | 7.45 |
| Citrus Fibers | ACF and 35% | | | | | | | | | | | | |
| and CS11 | CS11 | | | | | | | | | | | | |
| Scleroglucan | scleroglucan | | | | | | | | | | | | |
| | 65% CitriTex- | 76.63 | 77.11 | 76.75 | 76.83 | 0.22 | 0.18 | 0.19 | 0.20 | 9.45 | 9.44 | 9.49 | 9.46 |
| | ACF and 35% | | | | | | | | | | | | |
| | CS11 | | | | | | | | | | | | |
| | scleroglucan | | | | | | | | | | | | |
| | 65% CitriTex- | 72.65 | 71.74 | 72.18 | 72.19 | −0.54 | −0.46 | −0.47 | −0.49 | 5.75 | 6.09 | 5.98 | 5.94 |
| | ACF and 35% | | | | | | | | | | | | |
| | CS11 | | | | | | | | | | | | |
| | scleroglucan | | | | | | | | | | | | |
| | 65% CitriTex- | 73.35 | 73.15 | 73.00 | 73.17 | −0.15 | −0.07 | −0.08 | −0.10 | 7.57 | 7.63 | 7.73 | 7.64 |
| | ACF and 35% | | | | | | | | | | | | |
| | CS11 | | | | | | | | | | | | |
| | scleroglucan | | | | | | | | | | | | |
| | 65% CitriTex- | 78.80 | 78.86 | 78.87 | 78.84 | −0.31 | −0.30 | −0.29 | −0.30 | 5.26 | 5.27 | 5.26 | 5.26 |
| | ACF and 35% | | | | | | | | | | | | |
| | CS11 | | | | | | | | | | | | |
| | scleroglucan | | | | | | | | | | | | |
| | 65% CitriTex- | 80.63 | 81.60 | 80.95 | 81.06 | −0.26 | −0.31 | −0.25 | −0.27 | 5.18 | 5.17 | 5.44 | 5.26 |
| | ACF and 35% | | | | | | | | | | | | |
| | CS11 | | | | | | | | | | | | |
| | scleroglucan | | | | | | | | | | | | |
| | 65% CitriTex- | 81.39 | 81.22 | 81.50 | 81.37 | −0.31 | −0.30 | −0.33 | −0.31 | 5.70 | 5.66 | 5.69 | 5.68 |
| | ACF and 35% | | | | | | | | | | | | |
| | CS11 | | | | | | | | | | | | |
| | scleroglucan | | | | | | | | | | | | |
| | 65% CitriTex- | 79.80 | 79.50 | 79.72 | 79.67 | −0.51 | −0.49 | −0.50 | −0.50 | 4.36 | 4.51 | 4.42 | 4.43 |
| | ACF and 35% | | | | | | | | | | | | |
| | CS11 | | | | | | | | | | | | |
| | scleroglucan | | | | | | | | | | | | |
| | 65% CitriTex- | 81.69 | 81.58 | 81.59 | 81.62 | −0.42 | −0.38 | −0.41 | −0.40 | 4.71 | 4.75 | 4.65 | 4.70 |
| | ACF and 35% | | | | | | | | | | | | |
| | CS11 | | | | | | | | | | | | |
| | scleroglucan | | | | | | | | | | | | |
| | 65% CitriTex- | 82.82 | 82.43 | 82.28 | 82.51 | −0.42 | −0.42 | −0.40 | −0.41 | 4.68 | 4.67 | 4.74 | 4.70 |
| | ACF and 35% | | | | | | | | | | | | |
| | CS11 | | | | | | | | | | | | |
| | scleroglucan | | | | | | | | | | | | |
| | 65% CitriTex- | 78.62 | 78.65 | | 78.64 | −0.60 | −0.60 | | −0.60 | 3.59 | 3.56 | | 3.58 |
| | ACF and 35% | | | | | | | | | | | | |
| | CS11 | | | | | | | | | | | | |
| | scleroglucan | | | | | | | | | | | | |
| | 75% CitriTex- | 67.03 | 66.43 | 65.99 | 66.48 | −0.99 | −1.01 | −1.09 | −1.03 | 4.83 | 4.62 | 4.45 | 4.63 |
| | ACF and 25% | | | | | | | | | | | | |
| | CS11 | | | | | | | | | | | | |
| | scleroglucan | | | | | | | | | | | | |
| | 75% CitriTex- | 70.89 | 70.94 | 70.79 | 70.87 | −0.89 | −0.88 | −0.84 | −0.87 | 5.56 | 5.41 | 5.51 | 5.49 |
| | ACF and 25% | | | | | | | | | | | | |
| | CS11 | | | | | | | | | | | | |
| | scleroglucan | | | | | | | | | | | | |
| | 75% CitriTex- | 69.11 | 68.87 | 68.39 | 68.79 | −0.95 | −0.91 | −0.94 | −0.93 | 5.03 | 5.27 | 5.27 | 5.19 |
| | ACF and 25% | | | | | | | | | | | | |
| | CS11 | | | | | | | | | | | | |
| | scleroglucan | | | | | | | | | | | | |
| | 75% CitriTex- | 69.36 | 69.45 | 68.93 | 69.25 | −0.36 | −0.40 | −0.44 | −0.40 | 7.40 | 7.04 | 7.18 | 7.21 |
| | ACF and 25% | | | | | | | | | | | | |
| | CS11 | | | | | | | | | | | | |
| | scleroglucan | | | | | | | | | | | | |
| | 75% CitriTex- | 66.90 | 66.08 | 66.15 | 66.38 | −0.48 | −0.50 | −0.47 | −0.48 | 7.03 | 7.11 | 7.03 | 7.06 |
| | ACF and 25% | | | | | | | | | | | | |
| | CS11 | | | | | | | | | | | | |
| | scleroglucan | | | | | | | | | | | | |
| | 75% CitriTex- | 72.26 | 72.00 | 72.00 | 72.09 | −0.14 | −0.14 | −0.12 | −0.13 | 7.86 | 7.83 | 7.82 | 7.84 |
| | ACF and 25% | | | | | | | | | | | | |
| | CS11 | | | | | | | | | | | | |
| | scleroglucan | | | | | | | | | | | | |
| Co-processed | 65% CitriTex- | 79.00 | 79.09 | 79.02 | 79.04 | −0.09 | −0.13 | −0.09 | −0.10 | 7.75 | 7.53 | 7.75 | 7.68 |
| Citrus Fibers | ACF and 35% | | | | | | | | | | | | |
| and CS6 | CS6 | | | | | | | | | | | | |
| Scleroglucan | scleroglucan | | | | | | | | | | | | |
| | 65% CitriTex- | 79.07 | 78.98 | 78.92 | 78.99 | 0.10 | 0.07 | 0.03 | 0.07 | 8.67 | 8.54 | 8.45 | 8.55 |
| | ACF and 35% | | | | | | | | | | | | |
| | CS6 | | | | | | | | | | | | |
| | scleroglucan | | | | | | | | | | | | |
| | 65% CitriTex- | 75.21 | 75.15 | 75.01 | 75.12 | −0.30 | −0.30 | −0.31 | −0.30 | 7.02 | 6.86 | 6.93 | 6.94 |
| | ACF and 35% | | | | | | | | | | | | |
| | CS6 | | | | | | | | | | | | |
| | scleroglucan | | | | | | | | | | | | |
| | 65% CitriTex- | 74.72 | 74.78 | 74.65 | 74.72 | −0.09 | −0.09 | −0.08 | −0.09 | 7.94 | 7.98 | 7.92 | 7.95 |
| | ACF and 35% | | | | | | | | | | | | |
| | CS6 | | | | | | | | | | | | |
| | scleroglucan | | | | | | | | | | | | |
| COMPARTITIVE - | 90% CitriTex- | 69.43 | 69.32 | 69.34 | 69.36 | −0.72 | −0.70 | −0.73 | −0.72 | 6.43 | 6.46 | 6.41 | 6.43 |
| Co-processed | ACF and 10% | | | | | | | | | | | | |
| Citrus Fibers | Satiagel VPC | | | | | | | | | | | | |
| and Satiagel | 512 | | | | | | | | | | | | |
| | 75% CitriTex- | 66.56 | 66.50 | 66.65 | 66.57 | −0.87 | −0.87 | −0.87 | −0.87 | 5.27 | 5.25 | 5.30 | 5.27 |
| | ACF and 25% | | | | | | | | | | | | |
| | Satiagel VPC | | | | | | | | | | | | |
| | 512 | | | | | | | | | | | | |
| | 75% CitriTex- | 71.69 | 71.97 | 71.53 | 71.73 | −0.72 | −0.70 | −0.71 | −0.71 | 6.05 | 5.75 | 6.12 | 5.97 |
| | ACF and 25% | | | | | | | | | | | | |
| | Satiagel VPC | | | | | | | | | | | | |
| | 512 | | | | | | | | | | | | |
| | 75% CitriTex- | 66.20 | 66.03 | 66.12 | 66.12 | −0.45 | −0.56 | −0.49 | −0.50 | 7.24 | 7.01 | 7.16 | 7.14 |
| | ACF and 25% | | | | | | | | | | | | |
| | Satiagel VPC | | | | | | | | | | | | |
| | 512 | | | | | | | | | | | | |
| | 75% CitriTex- | 68.98 | 69.05 | 68.85 | 68.96 | −0.36 | −0.30 | −0.33 | −0.33 | 7.41 | 7.59 | 7.66 | 7.55 |
| | ACF and 25% | | | | | | | | | | | | |
| | Satiagel VPC | | | | | | | | | | | | |
| | 512 | | | | | | | | | | | | |
| | 75% CitriTex- | 71.67 | 71.65 | 71.68 | 71.67 | −0.24 | −0.25 | −0.17 | −0.22 | 7.87 | 7.77 | 7.94 | 7.86 |
| | ACF and 25% | | | | | | | | | | | | |
| | Satiagel VPC | | | | | | | | | | | | |
| | 512 | | | | | | | | | | | | |
| | 75% CitriTex- | 70.90 | 70.86 | 70.88 | 70.88 | −0.69 | −0.63 | −0.63 | −0.65 | 5.93 | 5.94 | 6.01 | 5.96 |
| | ACF and 25% | | | | | | | | | | | | |
| | Satiagel VPC | | | | | | | | | | | | |
| | 512 | | | | | | | | | | | | |
| | 65% CitriTex- | 69.91 | 69.85 | 69.82 | 69.86 | −0.39 | −0.36 | −0.36 | −0.37 | 7.19 | 7.24 | 7.29 | 7.24 |
| | ACF and 35% | | | | | | | | | | | | |
| | Satiagel VPC | | | | | | | | | | | | |
| | 512 | | | | | | | | | | | | |
| Control - | Citri-Tex ACF | 69.88 | 69.67 | 69.60 | 69.72 | 0.13 | 0.07 | −0.01 | 0.06 | 9.08 | 8.80 | 8.80 | 8.89 |
| Citrus Fibers | | | | | | | | | | | | | |
| Control - | CS11 | 75.30 | 74.98 | 76.71 | 75.66 | −0.87 | 0.92 | −0.76 | −0.24 | 4.79 | 4.81 | 4.81 | 4.80 |
| Scleroglucan | Scleroglucan | | | | | | | | | | | | |
| | CS11 | 78.74 | 78.20 | 76.33 | 77.76 | −0.63 | −0.64 | −0.73 | −0.67 | 1.13 | 1.07 | 1.01 | 1.07 |
| | Scleroglucan | | | | | | | | | | | | |
| Control - | CS6 | 81.38 | 81.74 | 81.51 | 81.54 | 0.54 | 0.58 | 0.55 | 0.56 | 4.79 | 4.81 | 4.81 | 4.80 |
| Scleroglucan | Scleroglucan | | | | | | | | | | | | |
| | CS6 | 76.20 | 76.23 | 76.02 | 76.15 | −0.78 | −0.79 | −0.78 | −0.78 | 3.67 | 3.65 | 3.74 | 3.69 |
| | Scleroglucan | | | | | | | | | | | | |

Table 5 shows a topical formulation comprising a ratio of 35:65 CS11 scleroglucan to citrus fibers has a smooth, fresh texture with fast absorption into the skin.

Table 6 shows that topical formulations prepared with emulsion comprising citrus fibers and scleroglucan have a white or off-white colour.

Furthermore, table 6 shows emulsions comprising either the citrus fibers alone or the citrus fibers with other compounds produce less desirable beige, grey and dark topical formulations.

TABLE 6
Example Formulations

| Trade Name | INCI Name | Supplier | Ex 2 | Ex 3 | Ex 4 | Gel Ex 5 |
|---|---|---|---|---|---|---|
| Aqua | Aqua | — | q.s.p = 100 | q.s.p = 100 | q.s.p = 100 | q.s.p = 100 |
| C*HiForm A | Hydroxypropyl | Cargill | | | 1.5% | |
| 12747 | Starch Phosphate | | | | | |
| Glycerine | Glycerine | Cargill | 1% | | | 1% |
| Zerose 16952 | Erythritol | Cargill | 1% | | | 1% |
| Dermofeel PA-3 | Sodium Phytate | Dr. | | | 0.10% | |
| | (and) Aqua (and) | Straetmans | | | | |
| | Alcohol | | | | | |
| CitriTex-ACF + | Citrus Peel Fibers | Cargill | 1% | 2% | 1.5% | 2% |
| Actigum CS11 | (and) Sclerotium | | | | | |
| | Gum | | | | | |
| Agri-pure AP | Brassica | Cargill | 10% | | 30% | |
| 75-R | Campestris Seed | | | | | |
| | Oil | | | | | |
| Agri-pure AP | Hydrogenated | Cargill | | 20% | | |
| 620 | Coconut Oil | | | | | |
| Emultop | Lecithin | Cargill | | | 0.3% | 0.3% |
| Velvet IP | | | | | | |
| Iscaguard | Phenoxyethanol | Isca | 1% | 1% | 1% | 1% |
| PEGH | (and) | | | | | |
| | Ethylhexylglycerin | | | | | |
| Tocopheryl | Tocopheryl | Rita | | | 0.20% | |
| Acetate | Acetate | | | | | |
| Fragrance | Fragrance | Fragrance | qs | qs | qs | qs |

### Example 3

Shampoos were prepared with co-processed citrus fibers and scleroglucan according to the invention. For comparative purposes, shampoo formulation 3 did not include co-processed citrus fiber and scleroglucan. The shampoo formulations are detailed in Table 7.

TABLE 7
Shampoo Formulations

| | Product | | |
|---|---|---|---|
| | Shampoo 1 | Shampoo 2 | Shampoo 3 |
| | (% W active) | (% W active) | (% W active) |
| Aqua | q.s.p = 100 | q.s.p = 100 | q.s.p = 100 |
| Co-processed Citrus | 0.4% | 0.4% | — |
| Fiber with Scleroglucan | | | |
| Sodium Lauryl ether | 12% | 12% | 12% |
| sulfate | | | |
| Cocamidopropyl Betaine | 2% | 2% | 2% |
| Silicone | — | 1.5% | — |
| Guar | 0.2% | 0.2% | 0.2% |
| Hydroxypropyltrimonium | | | |
| Chloride (e;g; activsoft | | | |
| C-17, from Innospec) | | | |
| Sunshine Glitter white | 0.2% | 0.2% | 0.2% |
| (e.g. CB03400, | | | |
| SunChemical) | | | |
| Salt (NaCl) | 1.5% | 1.5% | 1.5% |
| Citric Acid or/and | pH | pH | pH |
| NaOH | adjustment | adjustment | adjustment |
| Phenoxyethanol (and) | 1% | 1% | 1% |
| Ethylhexylglycerin (e.g. | | | |
| Isaguard PEHG, from | | | |
| Isca) | | | |
| Perfume | qs | qs | qs |

Shampoo formulations 1 and 2 displayed good stability with no separation between the oil phase and aqueous phase.

In contrast, shampoo formulation 3 did not include co-processed citrus fibers and scleroglucan and displayed significant signs of instability at room temperature, with the suspended glitter particles gradually separating down from the formulation.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

Although certain example embodiments of the invention have been described, the scope of the appended claims is not intended to be limited solely to these embodiments. The claims are to be construed literally, purposively, and/or to encompass equivalents.

1. An emulsion comprising an aqueous phase and an oil phase, the emulsion comprising citrus fibers and a 1,3-β-D-glucan.

2. An emulsion according to claim 1, wherein the ratio of citrus fibers to 1,3-β-D-glucan is selected from the group consisting of: between 90:10 and 10:90, between 80:20 and 20:80, between 70:30 and 30:70, between 60:40 and 40:60, and 50:50.

3. An emulsion according to claim 2, wherein the amount of aqueous phase in the emulsion is between 70 wt % to 95 wt %.

4. An emulsion according to claim 3, wherein the amount of oil phase in the emulsion is between 0.1 wt % to 30 wt %.

5. An emulsion according to claim 1, wherein the citrus fibers and/or 1,3-β-D-glucan are dispersed in the aqueous phase, the oil phase, or both.

6. An emulsion according to claim 4, wherein the oil phase comprises a natural oil, hydrogenated oil, triglyceride, rapeseed oil, or non-natural oil.

7. An emulsion according to claim 1, further comprising at least one further ingredient selected from the group consisting of preservative, salt, vitamin, emulsifier, texturiser, nutrient, micronutrient, sugar, protein, polysaccharide, polyol, glucose, sucrose, glycerol, sorbitol, pH adjusters, emollients, dyes, pigments, skin actives, waxes or silicones.

8. An emulsion according to claim 2, wherein the citrus fibers and 1,3-β-D-glucan when used to manufacture the emulsion are in the form of a blend or co-processed blend.

9. An emulsion according to claim 8, wherein the amount of the blend in the emulsion is selected from the group consisting of: between 0.1 wt % to 5 wt %, between 0.2 wt % to 3 wt %, and between 0.3 wt % to 3 wt %.

10. (canceled)

11. A topical formulation comprising the emulsion of claim 9.

12. The topical formulation of claim 11, that is a cosmetic, cream, balm, soap, sunscreen, moisturizer, lotion, shampoo, hair styling product, leave on hair gel, conditioner, hair care product, scalp treatment, or skin treatment.

13. A dry blend comprising citrus fibers and a 1,3-β-D-glucan, wherein the dry blend is a co-processed blend.

14. A dry blend according to claim 13, wherein the concentration of the citrus fibers in the blend is selected from the group consisting of: between 25 wt % and 99 wt %, between 35 wt % and 95 wt %, and between 55 wt % and 85 wt %; and the concentration of the 1,3-β-D-glucan in the blend is selected from the group consisting of between 0.1 wt % to 75 wt %, between 1 wt % and 65 wt %, or between 5 wt % and 55 wt %, and between 10 wt % and 55 wt %.

15. An dry blend according to claim 13, wherein the ratio of citrus fibers to 1,3-β-D-glucan is selected from the group consisting of between 90:10 and 10:90, between 80:20 and 20:80, between 70:30 and 30:70, between 60:40 and 40:60, and 50:50.

16. An aqueous mixture comprising the dry blend of claim 15 and wherein the viscosity of the aqueous mixture is from 3 to 300,000 Cps.

17. A topical formulation comprising the aqueous mixture of claim 16.

18. The topical formulation of claim 17 that is a personal care product.

19. A process for producing an emulsion of citrus fibers and a 1,3-β-D-glucan -comprising the steps of:
a) providing a blend of citrus fibers and a 1,3-β-D-glucan;
b) adding the blend to water and mixing to form an aqueous phase.
c) dispersing oil in the aqueous phase to obtain an emulsion, optionally, wherein the process further includes a homogenization step before and/or after step c).

20. The emulsion of claim 8, wherein the 1,3-β-D-glucan is scleroglucan.

21. (canceled)

22. (canceled)

23. The dry blend of claim 14, wherein the 1,3-β-D-glucan is scleroglucan.
